(19) United States
(12) Patent Application Publication
(10) Pub. No.: US 2025/0332416 A1
(43) Pub. Date: Oct. 30, 2025

(54) TRANSCUTANEOUS POWER TRANSFER

(71) Applicant: COCHLEAR LIMITED, Macquarie University, NSW (AU)

(72) Inventors: Helmut Christian EDER, Macquarie University (AU); Frédéric GODDEERIS, Macquarie University (AU)

(21) Appl. No.: 18/868,528

(22) PCT Filed: May 23, 2023
(86) PCT No.: PCT/IB2023/055304

§ 371 Date: Nov. 22, 2024

Related U.S. Application Data
(60) Provisional application No. 63/344,839, filed on May 23, 2022.

(51) Int. Cl. A61N 1/36; A61N 1/378

(52) U.S. Cl. CPC A61N 1/36038; A61N 1/3787

(57) ABSTRACT

A device including an external component of a prosthesis configured to provide power to an implanted device implanted in a human, wherein the external component is configured so that a level of power output to the implanted device is dynamically varied, based on data based on data that is based on a load of the implanted device, in a digital binary manner.

### CROSS-REFERENCE TO RELATED APPLICATIONS

This application claims priority to U.S. Provisional Application No. 63/344,839, entitled TRANSCUTANEOUS POWER TRANSFER, filed on May 23, 2022, naming Helmut Christian EDER as an inventor, the entire contents of that application being incorporated herein by reference in its entirety.

### BACKGROUND

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In an exemplary embodiment, there is a device that includes an external component of a prosthesis configured to provide power to an implanted device implanted in a human, wherein the external component is configured so that a level of power output to the implanted device is dynamically varied, based on data based on data that is based on a load of the implanted device, in a digital binary manner.

In an exemplary embodiment there is a device, comprising an external component of a prosthesis configured to provide power to an implanted device implanted in a human, wherein the external component is configured so that a principal power varying regime is based on varying power output to the implanted device by varying lengths of temporal periods of continuous maximum power output.

In an exemplary embodiment, there is a method comprising automatically obtaining data based on data that is influenced by a power load on a power consuming implanted medical device implanted in a human, automatically analyzing the obtained data, and transcutaneously providing power to the implanted medical device by increasing power to the implant to a maximum amount from a minimum amount or decreasing power to the implant to the minimum amount from the maximum amount depending on the result of the analysis.

In an embodiment, a cochlear implant external component, comprising a housing, a radio-frequency inductance coil connected to the housing or supported in the housing, the radio-frequency inductance coil configured to provide power to an implanted device implanted in a human, a battery and circuitry configured to provide power from the battery to the radio-frequency inductance coil, wherein the circuity is configured so that a level of power output to the implanted device is dynamically varied, based on data that is based on a load of the implanted device, in a digital binary manner.

### BRIEF DESCRIPTION OF THE DRAWINGS

Embodiments are described below with reference to the attached drawings, in which:

FIG. 1 is a perspective view of an exemplary hearing prosthesis;

FIG. 2 presents a functional block diagram of an exemplary cochlear implant;

FIG. 3A and FIGS. 3B and 3C present exemplary systems of communication between devices;

FIG. 4 presents an exemplary retinal prosthesis;

FIG. 5 presents an exemplary vestibular implant;

FIG. 6 presents exemplary data and power transfer;

FIG. 7 presents an exemplary RF envelop;

FIG. 8 presents an exemplary RF envelop according to an exemplary embodiment;

FIG. 9 presents an exemplary usage scenario according to an exemplary embodiment;

FIG. 9A presents another RF envelop (top portion) according to an exemplary embodiment;

FIG. 10 presents an exemplary target voltage scenario; and

FIG. 11 presents an exemplary algorithm according to an exemplary embodiment.

### DETAILED DESCRIPTION

Merely for ease of description, the techniques presented herein are described herein with reference by way of background to an illustrative medical device, namely a cochlear implant. However, it is to be appreciated that the techniques presented herein may also be used with a variety of other medical devices that, while providing a wide range of therapeutic benefits to recipients, patients, or other users, may benefit from setting changes based on the location of the medical device. For example, the techniques presented herein may be used to determine the viability of various types of prostheses, such as, for example, a vestibular implant and/or a retinal implant, with respect to a particular human being. And with regard to the latter, the techniques presented herein are also described with reference by way of background to another illustrative medical device, namely a retinal implant. The techniques presented herein are also applicable to the technology of vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation, etc.

And while the teachings detailed herein are directed towards stimulating tissue inside an inner ear of a human to evoke a hearing percept where the human cannot otherwise hear, it is noted that any disclosure herein with respect to a cochlear implant in general, and the nerves or tissue that is stimulated by the electrode array thereof, corresponds to a disclosure of an alternate embodiment with respect to an eye system in general, and the nerves thereof in particular, including the optic nerves, as well as a retinal implant/vision implant and/or a vestibular implant and/or the tissue that is stimulated by such device, such disclosure being made in the interest of textual economy.

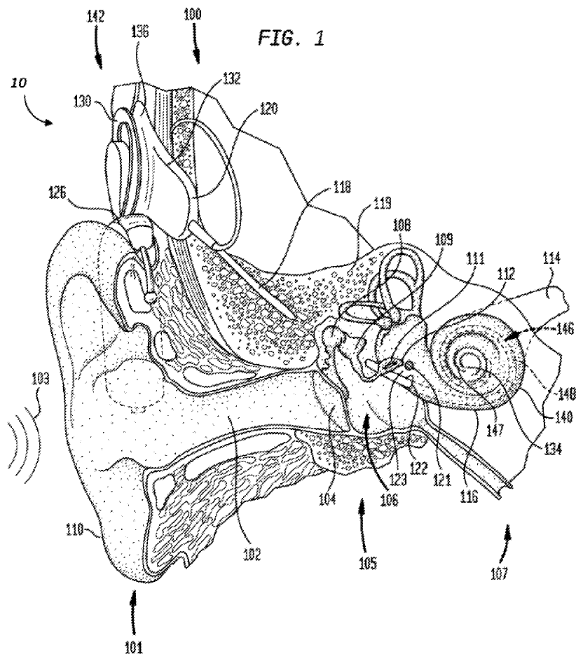

FIG. 1 is a perspective view of a cochlear implant, referred to as cochlear implant 100, implanted in a recipient, to which some embodiments detailed herein and/or variations thereof are applicable. Particularly, as will be detailed below, there are aspects of a cochlear implant that are utilized with respect to a vestibular implant, and thus there is utility in describing features of the cochlear implant for purposes of understanding a vestibular implant. The cochlear implant 100 is part of a system 10 that can include external components in some embodiments, as will be detailed below. Additionally, it is noted that the teachings detailed herein are also applicable to other types of hearing prostheses, such as, by way of example only and not by way of limitation, bone conduction devices (percutaneous, active transcutaneous and/or passive transcutaneous), direct acoustic cochlear stimulators, middle ear implants, and conventional hearing aids, etc. Indeed, it is noted that the teachings detailed herein are also applicable to so-called multi-mode devices. In an exemplary embodiment, these multi-mode devices apply both electrical stimulation and acoustic stimulation to the recipient. In an exemplary embodiment, these multi-mode devices evoke a hearing percept via electrical hearing and bone conduction hearing.

In view of the above, it is to be understood that at least some embodiments detailed herein and/or variations thereof are directed towards a body-worn sensory supplement medical device (e.g., the hearing prosthesis of FIG. 1, which supplements the hearing sense, even in instances when there are no natural hearing capabilities, for example, due to degeneration of previous natural hearing capability or to the lack of any natural hearing capability, for example, from birth). It is noted that at least some exemplary embodiments of some sensory supplement medical devices are directed towards devices such as middle ear implants or active transcutaneous bone conduction devices, which supplement the hearing sense in instances where some natural hearing capabilities have been retained, and visual prostheses (both those that are applicable to recipients having some natural vision capabilities and to recipients having no natural vision capabilities) all of which utilize transcutaneous power transfer. Accordingly, the teachings detailed herein are applicable to any type of sensory supplement medical device to which the teachings detailed herein are enabled for use therein in a utilitarian manner that utilizes transcutaneous power transfer. In this regard, the phrase sensory supplement medical device refers to any device that functions to provide sensation to a recipient irrespective of whether the applicable natural sense is only partially impaired or completely impaired, or indeed never existed.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear channel 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1 with an external device 142, that is part of system 10 (along with cochlear implant 100), which, as described below, is configured to provide power to the cochlear implant, where the implanted cochlear implant includes a battery that is recharged by the power provided from the external device 142.

In the illustrative arrangement of FIG. 1, external device 142 can comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1, the external energy transfer assembly comprises an external coil 130 that forms part of an inductive radio frequency (RF) communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1 is merely illustrative, and other external devices may be used with embodiments.

Cochlear implant 100 comprises an internal energy transfer assembly 132 which can be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate electrode assembly 118. In some embodiments, internal energy transfer assembly 132 and main implantable component 120 are hermetically sealed within a biocompatible housing. In some embodiments, main implantable component 120 includes an implantable microphone assembly (not shown) and a sound processing unit (not shown) to convert the sound signals received by the implantable microphone in internal energy transfer assembly 132 to data signals. That said, in some alternative embodiments, the implantable microphone assembly can be located in a separate implantable component (e.g., that has its own housing assembly, etc.) that is in signal communication with the main implantable component 120 (e.g., via leads or the like between the separate implantable component and the main implantable component 120). In at least some embodiments, the teachings detailed herein and/or variations thereof can be utilized with any type of implantable microphone arrangement.

Main implantable component 120 further includes a stimulator unit (also not shown) which generates electrical stimulation signals based on the data signals. The electrical stimulation signals are delivered to the recipient via elongate electrode assembly 118.

Elongate electrode assembly 118 has a proximal end connected to main implantable component 120, and a distal end implanted in cochlea 140. Electrode assembly 118 extends from main implantable component 120 to cochlea 140 through mastoid bone 119. In some embodiments electrode assembly 118 may be implanted at least in basal region 116, and sometimes further. For example, electrode assembly 118 may extend towards apical end of cochlea 140, referred to as cochlea apex 134. In certain circumstances, electrode assembly 118 may be inserted into cochlea 140 via a cochleostomy 122. In other circumstances, a cochleostomy may be formed through round window 121, oval window 112, the promontory 123 or through an apical turn 147 of cochlea 140.

Electrode assembly 118 comprises a longitudinally aligned and distally extending array 146 of electrodes 148, disposed along a length thereof. As noted, a stimulator unit generates stimulation signals which are applied by electrodes 148 to cochlea 140, thereby stimulating auditory nerve 114.

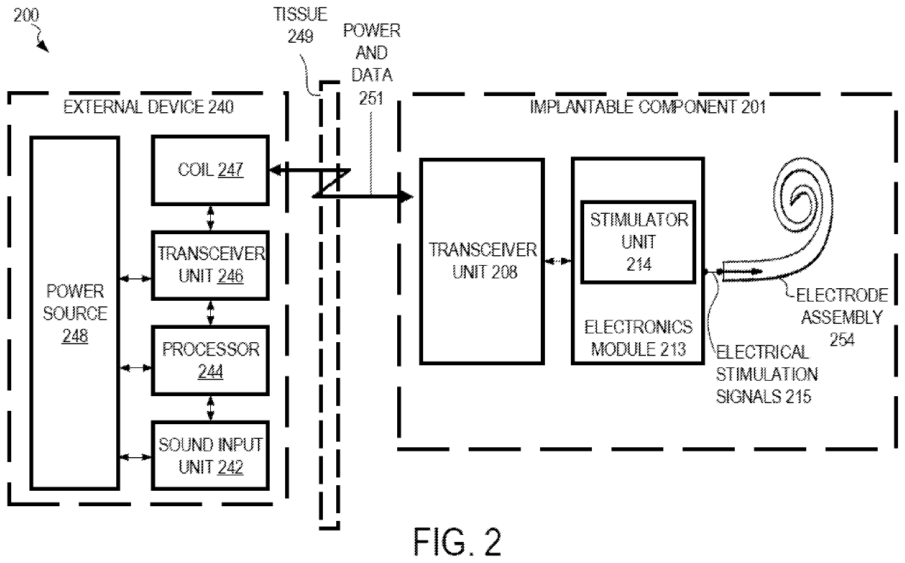

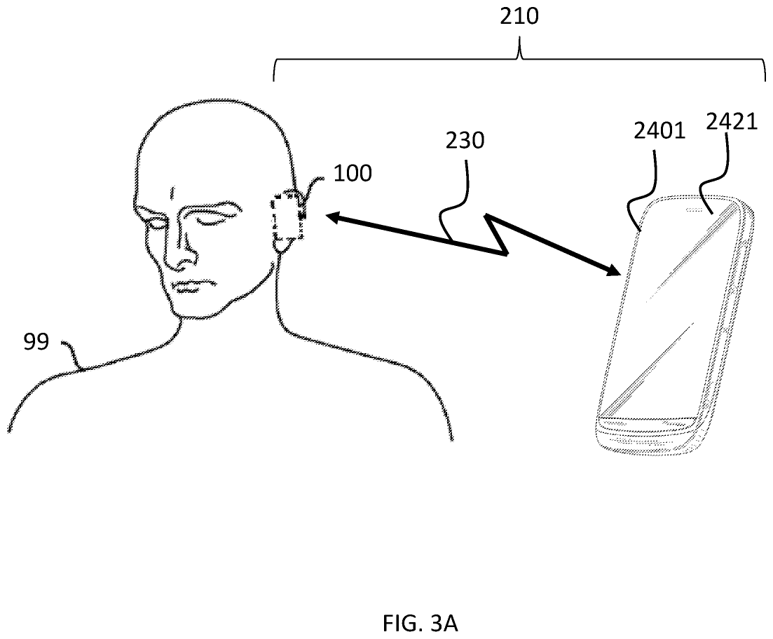

FIG. 2 is a functional block diagram of a cochlear implant system 200 in accordance with certain examples of the technology described herein. The cochlear implant system 200 includes an implantable component 201 (e.g., implantable component 100 of FIG. 1) configured to be implanted beneath a recipient's skin or other tissue 249, and an external device 240 (e.g., the external device 142 of FIG. 1).

The external device 240 can be configured as a wearable external device, such that the external device 240 is worn by a recipient in close proximity to the implantable component, which can enable the implantable component 201 to receive power and stimulation data from the external device 240. As described in FIG. 1, magnets can be used to facilitate an operational alignment of the external device 240 with the implantable component 201. With the external device 240 and implantable component 201 in close proximity, the transfer of power and data can be accomplished through the use of near-field electromagnetic radiation, and the components of the external device 240 can be configured for use with near-field electromagnetic radiation.

Implantable component 201 can include a transceiver unit 208, electronics module 213, which module can be a stimulator assembly of a cochlear implant, and an electrode assembly 254 (which can include an array of electrode contacts disposed on lead 118 of FIG. 1). The transceiver unit 208 is configured to transcutaneously receive power and/or data from external device 240. As used herein, transceiver unit 208 refers to any collection of one or more components which form part of a transcutaneous energy transfer system. Further, transceiver unit 208 can include or be coupled to one or more components that receive and/or transmit data or power. For example, the example includes a coil for a magnetic inductive arrangement coupled to the transceiver unit 208. Other arrangements are also possible, including an antenna for an alternative RF system, capacitive plates, or any other utilitarian arrangement. In an example, the data modulates the RF carrier or signal containing power. The transcutaneous communication link established by the transceiver unit 208 can use time interleaving of power and data on a single RF channel or band to transmit the power and data to the implantable component 201. In some examples, the processor 244 is configured to cause the transceiver unit 246 to interleave power and data signals, such as is described in U.S. Patent Publication Number 2009/0216296 to Meskens. In this manner, the data signal is modulated with the power signal, and a single coil can be used to transmit power and data to the implanted component 201. Various types of energy transfer, such as infrared (IR), electromagnetic, capacitive and inductive transfer, can be used to transfer the power and/or data from the external device 240 to the implantable component 201. Other types of power transfer systems separate the power signal from the data signal. The teachings herein can be used in both types of arrangements.

Aspects of the implantable component 201 can require a source of power to provide functionality, such as receive signals, process data, or deliver electrical stimulation. The source of power that directly powers the operation of the aspects of the implantable component 201 can be described as operational power. There are two exemplary ways that the implantable component 201 can receive operational power: a power source internal to the implantable component 201 (e.g., a battery) or a power source external to the implantable component. However, other approaches or combinations of approaches are possible. For example, the implantable component may have a battery but nonetheless receive operational power from the external component (e.g., to preserve internal battery life when the battery is sufficiently charged).

The internal power source can be a power storage element (not pictured). The power storage element can be configured for the long-term storage of power, and can include, for example, one or more rechargeable batteries. Power can be received from an external source, such as the external device 240, and stored in the power storage element for long-term use (e.g., charge a battery of the power storage element). The power storage element can then provide power to the other components of the implantable component 201 over time as needed for operation without needing an external power source. In this manner, the power from the external source may be considered charging power rather than operational power, because the power from the external power source is for charging the battery (which in turn provides operational power) rather than for directly powering aspects of the implantable component 201 that require power to operate. The power storage element can be a long-term power storage element configured to be a primary power source for the implantable component 201.

In some embodiments, the implantable component 201 receives operational power from the external device 240 and the implantable component 201 does not include an internal power source (e.g., a battery)/internal power storage device (but can include capacitors). In other words, the implantable component 201 is powered solely by the external device 240 or another external device, which provides enough power to the implantable component 201 to allow the implantable component to operate (e.g., receive data signals and take an action in response). The operational power can directly power functionality of the device rather than charging a power storage element of the external device implantable component 201. In these examples, the implantable component 201 can include incidental components that can store a charge (e.g., capacitors) or small amounts of power, such as a small battery for keeping volatile memory powered or powering a clock (e.g., motherboard CMOS batteries). But such incidental components would not have enough power on their own to allow the implantable component to provide primary functionality of the implantable component 201 (e.g., receiving data signals and taking an action in response thereto, such as providing stimulation) and therefore cannot be said to provide operational power even if they are integral to the operation of the implantable component 201.

As shown, electronics module 213 includes a stimulator unit 214 (e.g., which can correspond to the stimulator of FIG. 1). Electronics module 213 can also include one or more other components used to generate or control delivery of electrical stimulation signals 215 to the recipient. As described above with respect to FIG. 1, a lead (e.g., elongate lead 118 of FIG. 1) can be inserted into the recipient's cochlea. The lead can include an electrode assembly 254 configured to deliver electrical stimulation signals 215 generated by the stimulator unit 214 to the cochlea.

In the example system 200 depicted in FIG. 2, the external device 240 includes a sound input unit 242, a sound processor 244, a transceiver unit 246, a coil 247, and a power source 248. The sound input unit 242 is a unit configured to receive sound input. The sound input unit 242 can be configured as a microphone (e.g., arranged to output audio data that is representative of a surrounding sound environment), an electrical input (e.g., a receiver for a frequency modulation (FM) hearing system), and/or another component for receiving sound input. The sound input unit 242 can be or include a mixer for mixing multiple sound inputs together.

The processor 244 is a processor configured to control one or more aspects of the system 200, including converting sound signals received from sound input unit 242 into data signals and causing the transceiver unit 246 to transmit power and/or data signals. The transceiver unit 246 can be configured to send or receive power and/or data 251. For example, the transceiver unit 246 can include circuit components that send power and data (e.g., inductively) via the coil 247. The data signals from the sound processor 244 can be transmitted, using the transceiver unit 246, to the implantable component 201 for use in providing stimulation or other medical functionality.

The transceiver unit 246 can include one or more antennas or coils for transmitting the power or data signal, such as coil 247. The coil 247 can be a wire antenna coil having of multiple turns of electrically insulated single-strand or multi-strand wire. The electrical insulation of the coil 247 can be provided by a flexible silicone molding. Various types of energy transfer, such as infrared (IR), radiofrequency (RF), electromagnetic, capacitive and inductive transfer, can be used to transfer the power and/or data from external device 240 to implantable component 201.

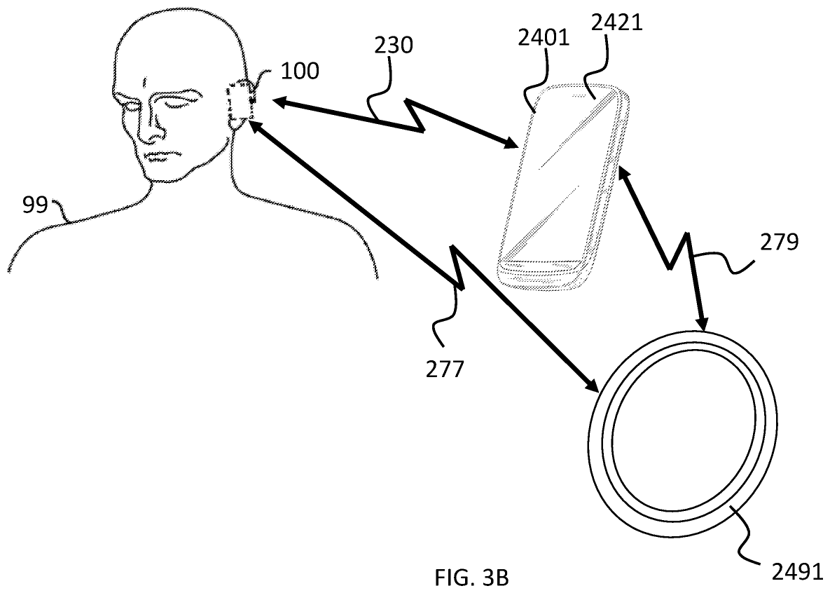

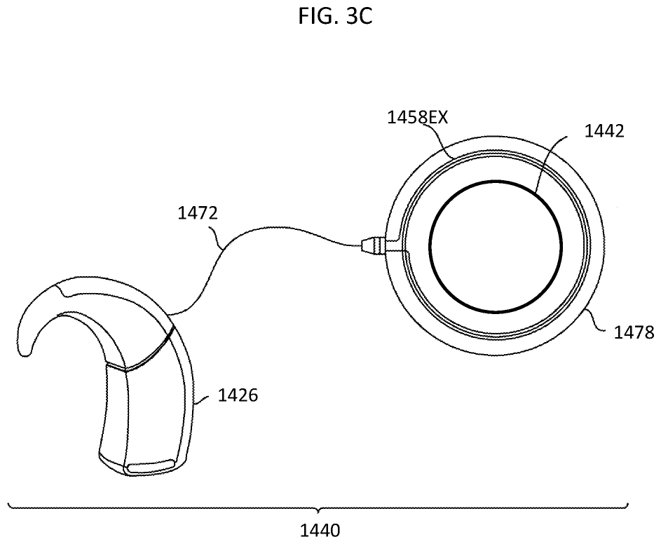

FIG. 3A depicts an exemplary system 210 according to an exemplary embodiment, including hearing prosthesis 100, which, in an exemplary embodiment, corresponds to cochlear implant 100 detailed above, and a portable body carried device (e.g., a portable handheld device as seen in FIG. 2A, a watch, a pocket device, etc.) 2401 in the form of a mobile computer having a display 2421. The system includes a wireless link 230 between the portable handheld device 2401 and the hearing prosthesis 100. In an embodiment, the prosthesis 100 is an implant implanted in recipient 99 (represented functionally by the dashed lines of box 100 in FIG. 3A).

In an exemplary embodiment, the system 210 is configured such that the hearing prosthesis 100 and the portable handheld device 2401 have a symbiotic relationship. In an exemplary embodiment, the symbiotic relationship is the ability to display data relating to, and, in at least some instances, the ability to control, one or more functionalities of the hearing prosthesis 100. In an exemplary embodiment, this can be achieved via the ability of the handheld device 2401 to receive data from the hearing prosthesis 100 via the wireless link 230 (although in other exemplary embodiments, other types of links, such as by way of example, a wired link, can be utilized). As will also be detailed below, this can be achieved via communication with a geographically remote device in communication with the hearing prosthesis 100 and/or the portable handheld device 2401 via link, such as by way of example only and not by way of limitation, an Internet connection or a cell phone connection. In some such exemplary embodiments, the system 210 can further include the geographically remote apparatus as well. Again, additional examples of this will be described in greater detail below.

As noted above, in an exemplary embodiment, the portable handheld device 2401 comprises a mobile computer and a display 2421. In an exemplary embodiment, the display 2421 is a touchscreen display. In an exemplary embodiment, the portable handheld device 2401 also has the functionality of a portable cellular telephone. In this regard, device 2401 can be, by way of example only and not by way of limitation, a smart phone, as that phrase is utilized generically. That is, in an exemplary embodiment, portable handheld device 2401 comprises a smart phone, again as that term is utilized generically.

It is noted that in some other embodiments, the device 2401 need not be a computer device, etc. It can be a lower tech recorder, or any device that can enable the teachings herein.

The phrase “mobile computer” entails a device configured to enable human-computer interaction, where the computer is expected to be transported away from a stationary location during normal use. Again, in an exemplary embodiment, the portable handheld device 2401 is a smart phone as that term is generically utilized. However, in other embodiments, less sophisticated (or more sophisticated) mobile computing devices can be utilized to implement the teachings detailed herein and/or variations thereof. Any device, system, and/or method that can enable the teachings detailed herein and/or variations thereof to be practiced can be utilized in at least some embodiments. (As will be detailed below, in some instances, device 2401 is not a mobile computer, but instead a remote device (remote from the hearing prosthesis 100. Some of these embodiments will be described below).)

In an exemplary embodiment, the portable handheld device 2401 is configured to receive data from a hearing prosthesis and present an interface display on the display from among a plurality of different interface displays based on the received data. Exemplary embodiments will sometimes be described in terms of data received from the hearing prosthesis 100. However, it is noted that any disclosure that is also applicable to data sent to the hearing prosthesis from the handheld device 2401 is also encompassed by such disclosure, unless otherwise specified or otherwise incompatible with the pertinent technology (and vice versa).

It is noted that in some embodiments, the system 210 is configured such that cochlear implant 100 and the portable device 2401 have a relationship. By way of example only and not by way of limitation, in an exemplary embodiment, the relationship is the ability of the device 2401 to serve as a remote microphone for the prosthesis 100 via the wireless link 230. Thus, device 2401 can be a remote mic. That said, in an alternate embodiment, the device 2401 is a stand-alone recording/sound capture device.

It is noted that in at least some exemplary embodiments, the device 2401 corresponds to an Apple Watch™ Series 1 or Series 2, as is available in the United States of America for commercial purchase as of Jan. 10, 2021. In an exemplary embodiment, the device 2401 corresponds to a Samsung Galaxy Gear™ Gear 2, as is available in the United States of America for commercial purchase as of Jan. 10, 2021. The device is programmed and configured to communicate with the prosthesis and/or to function to enable the teachings detailed herein.

In an exemplary embodiment, a telecommunication infrastructure can be in communication with the hearing prosthesis 100 and/or the device 2401. By way of example only and not by way of limitation, a telecoil 2491 or some other communication system (Bluetooth, etc.) is used to communicate with the prosthesis and/or the remote device. FIG. 2B depicts an exemplary quasi-functional schematic depicting communication between an external communication system 2491 (e.g., a telecoil), and the hearing prosthesis 100 and/or the handheld device 2401 by way of links 277 and 279, respectively (note that FIG. 3B depicts two-way communication between the hearing prosthesis 100 and the external audio source 2491, and between the handheld device and the external audio source 2491—in alternate embodiments, the communication is only one way (e.g., from the external audio source 2491 to the respective device)). It is noted that unless otherwise noted, the embodiment of FIG. 3B is applicable to any body worn medical device/implanted device disclosed herein in some embodiments.

FIG. 3C depicts an exemplary external component 1440. External component 1440 can correspond to external component 142 of the system 10 (it can also represent other body worn devices herein/devices that are used with implanted portions). As can be seen, external component 1440 includes a behind-the-ear (BTE) device 1426 which is connected via cable 1472 to an exemplary headpiece 1478 including an external inductance coil 1458EX, corresponding to the external coil of FIG. 1. As illustrated, the external component 1440 comprises the headpiece 1478 that includes the coil 1458EX and a magnet 1442. This magnet 1442 interacts with the implanted magnet (or implanted magnetic material) of the implantable component to hold the headpiece 1478 against the skin of the recipient. In an exemplary embodiment, the external component 1440 is configured to transmit and/or receive magnetic data and/or transmit power transcutaneously via coil 1458EX to the implantable component, which includes an inductance coil. The coil 1458X is electrically coupled to BTE device 1426 via cable 1472. BTE device 1426 may include, for example, at least some of the components of the external devices/components described herein.

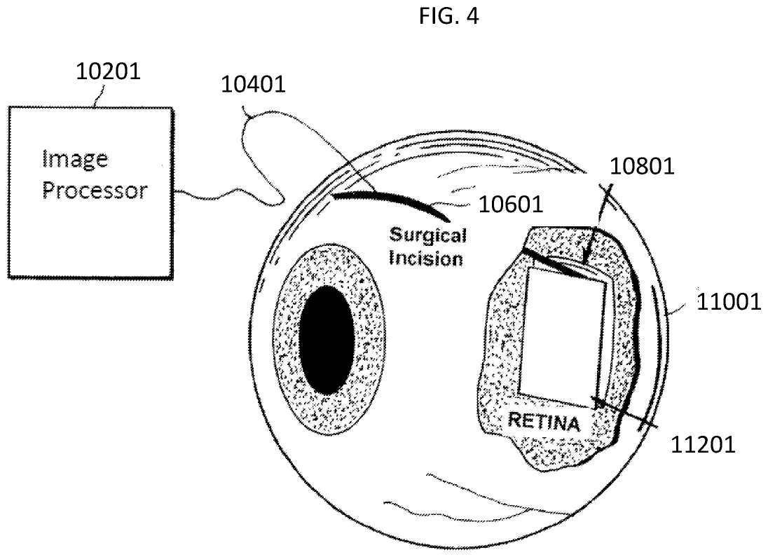

FIG. 4 presents an exemplary embodiment of a neural prosthesis in general, and a retinal prosthesis and an environment of use thereof, in particular, the components of which can be used in whole or in part, in some of the teachings herein. In some embodiments of a retinal prosthesis, a retinal prosthesis sensor-stimulator 10801 is positioned proximate the retina 11001. In an exemplary embodiment, photons entering the eye are absorbed by a microelectronic array of the sensor-stimulator 10801 that is hybridized to a glass piece 11201 containing, for example, an embedded array of microwires. The glass can have a curved surface that conforms to the inner radius of the retina. The sensor-stimulator 108 can include a microelectronic imaging device that can be made of thin silicon containing integrated circuitry that convert the incident photons to an electronic charge.

An image processor 10201 is in signal communication with the sensor-stimulator 10801 via cable 10401 which extends through surgical incision 00601 through the eye wall (although in other embodiments, the image processor 10201 is in wireless communication with the sensor-stimulator 10801). The image processor 10201 processes the input into the sensor-stimulator 10801 and provides control signals back to the sensor-stimulator 10801 so the device can provide processed output to the optic nerve. That said, in an alternate embodiment, the processing is executed by a component proximate with or integrated with the sensor-stimulator 10801. The electric charge resulting from the conversion of the incident photons is converted to a proportional amount of electronic current which is input to a nearby retinal cell layer. The cells fire and a signal is sent to the optic nerve, thus inducing a sight perception.

The retinal prosthesis can include an external device disposed in a Behind-The-Ear (BTE) unit or in a pair of eyeglasses, or any other type of component that can have utilitarian value. The retinal prosthesis can include an external light/image capture device (e.g., located in/on a BTE device or a pair of glasses, etc.), while, as noted above, in some embodiments, the sensor-stimulator 10801 captures light/images, which sensor-stimulator is implanted in the recipient.

In the interests of compact disclosure, any disclosure herein of a microphone or sound capture device corresponds to an analogous disclosure of a light/image capture device, such as a charge-coupled device. Corollary to this is that any disclosure herein of a stimulator unit which generates electrical stimulation signals or otherwise imparts energy to tissue to evoke a hearing percept corresponds to an analogous disclosure of a stimulator device for a retinal prosthesis. Any disclosure herein of a sound processor or processing of captured sounds or the like corresponds to an analogous disclosure of a light processor/image processor that has analogous functionality for a retinal prosthesis, and the processing of captured images in an analogous manner. Indeed, any disclosure herein of a device for a hearing prosthesis corresponds to a disclosure of a device for a retinal prosthesis having analogous functionality for a retinal prosthesis. Any disclosure herein of fitting a hearing prosthesis corresponds to a disclosure of fitting a retinal prosthesis using analogous actions. Any disclosure herein of a method of using or operating or otherwise working with a hearing prosthesis herein corresponds to a disclosure of using or operating or otherwise working with a retinal prosthesis in an analogous manner.

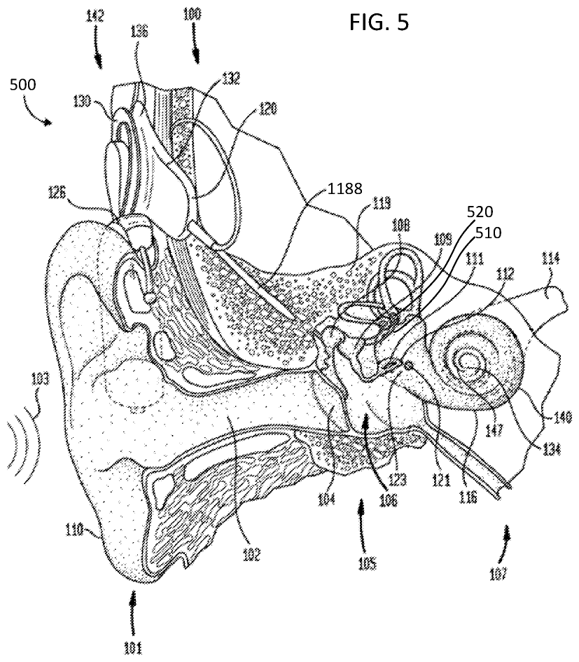

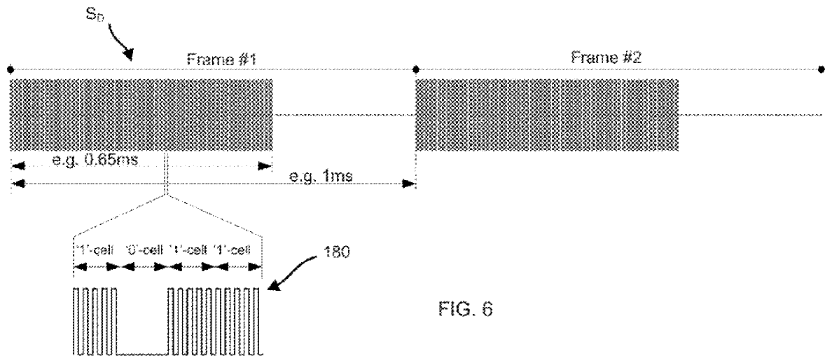

FIG. 5 depicts an exemplary vestibular implant 500. Some specific features are described utilizing the above noted cochlear implant of FIG. 1 in contacts for the various elements. In this regard, some features of a cochlear implant are utilized with vestibular implants. In the interest of textual and pictorial economy, various elements of the vestibular implant that generally correspond to the elements of the cochlear implant above are referenced utilizing the same numerals. Still, it is noted that some features of the vestibular implant 500 will be different from that of the cochlear implant above. By way of example only and not by way of limitation, there may not be a microphone on the behind-the-ear device 126. Alternatively, sensors that have utilitarian value in the vestibular implant can be contained in the BTE device 126. By way of example only and not by way of limitation, motion sensors can be located in BTE device 126. There also may not be a sound processor in the BTE device. Conversely, other types of processors, such as those that process data obtained from the sensors, will be present in the BTE device 126. Power sources, such as a battery, will also be included in the BTE device 126. Consistent with the BTE device of the cochlear implant of FIG. 1, a transmitter/transceiver will be located in the BTE device or otherwise in signal communication therewith.

The implantable component includes a receiver stimulator in a manner concomitant with the above cochlear implant. Here, vestibular stimulator comprises a main implantable component 120 and an elongate electrode assembly 1188 (where the elongate electrode assembly 1188 has some different features from the elongate electrode assembly 118 of the cochlear implant, some of which will be described shortly). In some embodiments, internal energy transfer assembly 132 and main implantable component 120 are hermetically sealed within a biocompatible housing. In some embodiments, main implantable component 120 includes a processing unit (not shown) to convert data obtained by sensors, which could be on board sensors implanted in the recipient, into data signals.

Main implantable component 120 further includes a stimulator unit (also not shown) which generates electrical stimulation signals based on the data signals. The electrical stimulation signals are delivered to the recipient via elongate electrode assembly 1188.

It is briefly noted that while the embodiment shown in FIG. 5 represents a partially implantable vestibular implant, embodiments can include a totally implantable vestibular implant, such as, where, for example, the motion sensors are located in the implantable portion, in a manner analogous to a cochlear implant.

Elongate electrode assembly 1188 has a proximal end connected to main implantable component 120, and extends through a hole in the mastoid 119, in a manner analogous to the elongate electrode assembly 118 of the cochlear implant, and includes a distal end that extends to the inner ear. In some embodiments, the distal portion of the electrode assembly 1188 includes a plurality of leads 510 that branch out away from the main body of the electrode assembly 118 to electrodes 520. Electrodes 520 can be placed at the base of the semicircular ducts as shown in FIG. 5. In an exemplary embodiment, one or more of these electrodes are placed in the vicinity of the vestibular nerve branches innervating the semicircular canals. In some embodiments, the electrodes are located external to the inner ear, while in other embodiments, the electrodes are inserted into the inner ear. Note also while this embodiment does not include an electrode array located in the cochlea, in other embodiments, one or more electrodes are located in the cochlea in a manner analogous to that of a cochlear implant.

A vestibular implant can have utilitarian value with respect to a human if the human has an at least partially functioning neural system in the vestibular system. Conversely, if the neural system in the vestibular system is completely non-functional, there will be little to no utilitarian value with respect to implanting a vestibular implant in the human. Embodiments include devices, systems, and methods that can enable the determination of whether or not a human's neural system in the vestibular system has sufficient functionality that the human can at least somewhat benefit from a vestibular implant. This can have utilitarian value with respect to avoiding a scenario where a vestibular implant is implanted in the human but the implant will have little to no utilitarian value because the neural system is not sufficiently functional. Corollary to this is that a retinal implant can have utilitarian value with respect to a human if the human has and at least partially functioning neural system of the vision system.

Accordingly, the teachings herein are directed towards evaluating if a human is suitable for a vestibular implant before being implanted. At least some teachings detailed herein can enable data to be obtained that, when analyzed, can provide indicia indicative of whether or not a human's peripheral vestibular response (a response from the vestibular nerve, for example) is present and/or is sufficient enough to render the human a candidate for the utilitarian outcome with respect to a vestibular implant. The data can be data indicative of a vestibular never function, at least some function. Still further, the teachings detailed herein are directed towards, in some other embodiments, evaluating if a human is suitable for a retinal implant before being implanted. At least some teachings detailed herein can enable data to be obtained that, when analyzed, can provide indicia indicative of whether or not a human's response to electrical stimulation in the eye or otherwise the response to electrical stimulation of the nerves of the optical system is present and/or is sufficient enough to render the human a candidate for the utilitarian outcome with respect to a retinal implant.

It is also noted that at least some exemplary embodiments are directed towards evaluating whether a semicircular canal implant can have utilitarian value with respect to a given human under testing. That is, at least some exemplary embodiments include devices systems and methods of screening humans for semicircular canal implants

Some embodiments utilize Galvanic Vestibular Stimulation (GVS)—monopolar and/or bipolar, placed unilaterally or bilaterally, to evoke a sensation of movement with the human's head and/or body in its entirety in a stationary position. In an exemplary embodiment the GVS is used for monolateral stimulation (but both “sides” can be stimulated, in a serial or spaced apart manner, providing that there is sufficient temporal spacing between the stimulations). Embodiments include providing a weak current (AC or DC, depending on the embodiment) across the mastoid processes. This current is utilized to excite one or more of the otoliths and semicircular canal afferents. Embodiments can include a continuous stimulation and/or a pulse stimulation and/or a sine wave. Noise stimulation can be used as well.

More specifically, in at least some exemplary embodiments, the aforementioned current is utilized to evoke a sensation of head roll around a naso-occipital axis, canals stimulation. In some exemplary embodiments, evoked torsional eye movement response to GVS is obtained, and otherwise captured, and evaluated. If sufficient movement is present, this can be indicative of a sufficiently functioning neural system of the vestibular system, thus indicating that the human can have utilitarian experience with respect to a vestibular implant.

Some exemplary embodiments utilize the GVS to excite the synapse between vestibular hair cells and the eighth nerve afferents. Embodiments herein can thus provide information regarding “neural” rather than “sensory” function. Humans with hair-cell damage (e.g., from ototoxic drug exposure) who have a preserved eighth nerve afferent function can have normal or even increased responses to galvanic vestibular stimulation despite absent responses to caloric and rotational testing.

The embodiments of FIGS. 1, 4 and 5 (and exemplary embodiments used in active transcutaneous bone conduction devices and/or middle ear implants that use a transcutaneous power/data link to power an implanted actuator) require power transfer through the skin of the recipient to power the implanted device. The amount of power needed to be transferred can be great, and the needed power can fluctuate by large amounts, rapidly (all of these phrases are relative of course—some specific examples will be described below).

FIG. 6 shows a non-limiting example of an electrical signal S_{D} applied to the transmitter coil of the external device to transfer power to the implantable component at a duty cycle of about 65%. In the example of FIG. 6, the duty cycle of a signal is generally considered to be a ratio of an On time to the total frame time or On and Off time. For example, in FIG. 6, the total frame time is 1 ms and the On time is 0.65 ms, which results in a 65% duty cycle. Further, FIG. 6 illustrates how data can be encoded in the electrical signal S_{D}, for example, using a five cycle per cell encoding. More particularly, within the On time of the signal S_{D}, binary one's and zero's can be encoded as shown in the enlarged portion 180. Further, the signal S_{D} need not be a square wave, as generally illustrated. Rather, each signal burst can be modulated using known techniques, such as on-off keying (OOK), frequency-shift keying (FSK), phase-shift keying (PSK), and the like, to transfer power and/or data over the transcutaneous link.

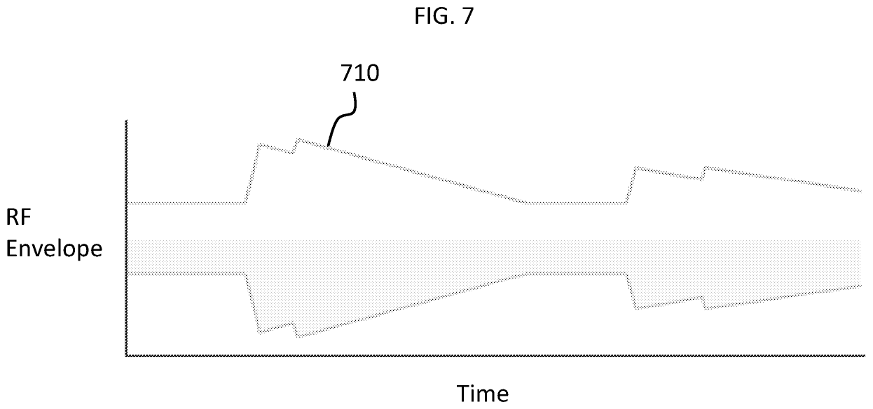

FIG. 7 represents a power level changing over 1 millisecond frames for about 150 milliseconds of a prior art transcutaneous power transfer system. The diagram of FIG. 7 shows the RF signal envelope (where the underlying signal is a sine wave). As can be seen, this prior art power regulation regime of FIG. 7 ramps up the external power as the load of the implant increases. This is known as attack (the increase of the power from the baseline level of power supplied to the implant (the horizontal lines depicted in FIG. 7 in this example)). Also as can be seen, this prior art power regulation regime of FIG. 7 ramps down the external power as the load of the implant decreases. This is known as decay. Note that the ramp up in the rent down need not be from and/or two, respectively, the baseline power provided as can be seen. In this example of the prior art transcutaneous power transfer system, the ramp ups and/or the ramp downs are provided in fixed power level steps based on the implant load otherwise the requirements of the implant.

In an exemplary embodiment of a prior art transcutaneous power transfer regime, the attack time to ramp up to the “needed” power transfer can be 50 or 100 or 150 ms or more. In an exemplary embodiment, the decay time to ramp down to the baseline power can be one or two or three seconds.

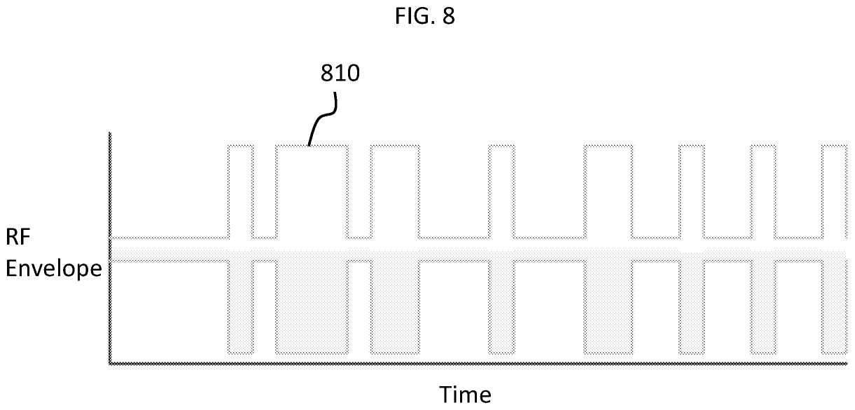

Embodiments of the present invention key off of the embodiment of FIG. 8, and are different from the example of FIG. 7 (although embodiments can use some of the features thereof, as will be detailed for example below—in an embodiment, unless otherwise specified, any of the structural features and/or method steps of the arrangement of FIG. 7 (or the embodiments of FIGS. 1-6 for that matter) can be used in the embodiments associated with FIG. 8 and onward and thereafter). FIG. 8 presents an exemplary embodiment that is different than that of FIG. 7. In this regard, the invention of this patent application corresponds to the embodiments of FIG. 8 and the figures thereafter. Any means-plus-function and/or step-plus function claims relating to the power transfer regime correspond to FIG. 8 and thereafter. But again, some exemplary embodiments of the invention can utilize some of the structure and/or function of the teachings detailed above, and the features thereof are not repeated below for the purposes of textual economy. And embodiments of the implants according to the invention can include one or more of the above noted structures and/or functions and/or can implement one or more of the above noted method actions can include methods that include one or more of the above noted method actions. However, with respect to the power transfer regime, the power transfer regime does not include the power transfer regime detailed above. This is at most related art that some aspects of the invention can utilize. In many embodiments, there are features that are distinctly different from the arrangement of FIG. 7.

FIG. 8 presents an RF Envelope vs. time for the present invention. In this embodiment, power transfer is ramped up and/or ramped down effectively immediately. Unlike the implant power regulation mechanism of FIG. 7, that ramps up and ramps down the external power in fixed power level steps based on the implant load, the transcutaneous power transfer regime FIG. 8 represents a regime that has a duty cycle implant power mechanism that delivers either maximum power or minimum power (in this exemplary embodiment—more on this below) depending on whether or not the load/needs of the implant have been met or otherwise are being met. In this arrangement, the external component meets the implant power needs by dynamically varying the percentage of time (e.g., duty cycle) of the maximum power RF frames relative to the minimum power RF frames. In this regard, FIG. 8 as compared to FIG. 7 provides a high level conceptual difference in the operating principals between the prior power transfer regime and the embodiment of the present invention that utilizes time on and time off of maximum duty cycle implant power regulation. In an embodiment, the device only uses the time on and time off for a period of time lasting at least any of the time periods herein and/or for at least 30, 35, 40, 45, 50, 55, 60, 65, 70, 75, 80, 85 or 90% or more of the time that the external device provides power to the implant during at least 1, 2, 3, 4, 5, 6, 7, 8, 9, 10, days or weeks or months.

In an exemplary embodiment of the transcutaneous power transfer regime of the present invention, the variation of the duty cycle in the binary manner detailed above regulates the voltage of the implant to meet a set target voltage of the implant. In an exemplary embodiment, the system implementing the transcutaneous power transfer regime according to the embodiments detailed herein continuously and/or periodically (in a sufficient time to enable the teachings detailed herein) evaluates the voltage of the implant to determine whether or not the implant voltage is above and/or below the target implant voltage. In an exemplary embodiment, the system is configured so that if a determination is made that the implant voltage is below the target voltage for the implant, the system applies maximum power for transfer from the external component to the implant. Conversely, again, in an exemplary embodiment, if the voltage of the implant is above the target voltage therefore, the system applies minimum power for transfer from the external component to the implant. As will be detailed below, the maximum and minimum power are but exemplary embodiments.

In an exemplary embodiment, the arrangement just described herein provides a system that effectively ensures automatic compensation for any load variations of the implant.

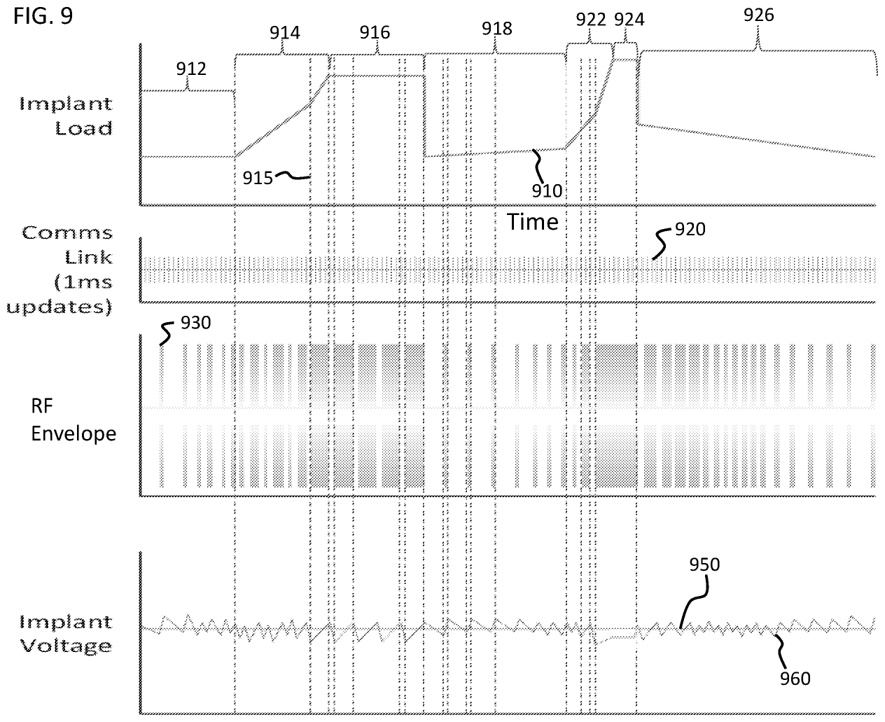

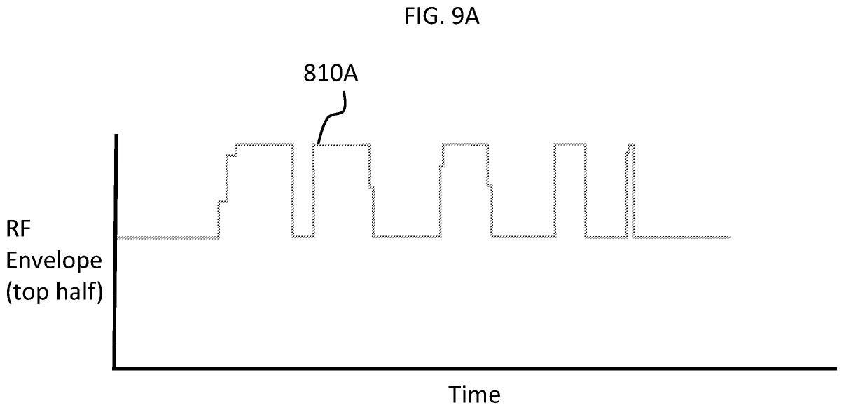

FIG. 9 presents four (4) charts showing respectively, implant load versus time, Communication Link data vs. time, and the RF power envelope vs. time for an exemplary scenario utilizing an exemplary embodiment of the teachings detailed herein. As seen with respect to the top chart, the implant load 910 varies over time, where the top chart depicts the milliwatts that are used by the implant over time. Briefly, in an exemplary embodiment, the target voltage of the implant is a voltage that has utilitarian value with respect to efficiency of operation of the implant, at least as it relates to charging of the implant. By way of example only and not by way of limitation, the target voltage can be a target voltage that has efficiency with respect to the overall operation of the system, such as by way of example, reducing the amount of heat generation from the implant, which represents waste with respect to energy transferred thereto, and thus represents a phenomenon that reduces the longevity of the battery of the external component relative to that which would be the case in the absence of this heat generation. The target voltage can be selected for any reason or a plurality of reasons. Embodiments include maintaining the target voltage above a certain level to avoid a reset under certain circumstances such as when a large transient occurs. In this exemplary embodiment, the target voltage is 5.5 V. In some embodiments, the target voltage can be higher than this or lower than this depending on the design or otherwise the anticipated performance requirements of the implant. In an exemplary embodiment, this is a fixed value, while in other embodiments, the system can be a system where the target voltage can be adjusted, either automatically by the system and/or under the control of a healthcare professional or the like. Some additional features associated with the target voltage and/or selection and/or control of the target voltage will be described below.

Still with reference to FIG. 9, as can be seen, there is a time interval number 912 where the implant load is constant. The time interval 912 spans a number of comms link updates 920 (here, by way of example, the interval 912 spans twenty (2) comms link updates, thus representing a 20 ms interval). By way of example only and not by way of limitation, the implant load during time interval 912 can be less than, equal to or greater than 2, 2.25, 2.5, 2.75, 3, 3.25, 3.5, 3.75, 4, 4.25, 4.5, 4.75, 5, 5.25, 5.5, 5.75, 6, 6.25, 6.5, 6.75, 7, 7.25, 7.5, 7.75, 8, 8.25, 8.5, 8.75, 9, 9.25, 9.5, 9.75, 10, 11, 12, 13, 14, 15, 16, 17, 18, 19, 20, 21, 22, 23, 24, 25, 26, 27, 28, 29, 30 mW, or any value or range of values therebetween in 0.01 mW increments (e.g., 2.28 mW, 7.88 mW, 3.03 to 5.55 mW, etc.). This can represent the load on the implant when the implant, in the case of a cochlear implant, is not evoking a hearing percept or otherwise when the recipient of the implant is in silence. This can correspond to the load that is required to keep the implant in a state of readiness to evoke a hearing percept or otherwise to provide stimulation to the recipient indicative of sound above the level of silence. This could be the minimum value to maintain the capacitors charged at a set level.

In an exemplary embodiment that utilizes a target voltage of the implant to control the power transferred to the implant, when the voltage of the implant falls below the target voltage for example, the system controls the external component to transfer maximum power to the implant. When the implant voltage meets or exceeds the target voltage, owing to the power transferred to the implant from the external component sufficient to address the current load conditions of the implant and/or because the load has been reduced for whatever reason, the system controls the external component to transfer the minimum power to the implant. This continues in an iterative manner for example during the use of the implant to evoke a hearing percept or otherwise during the time period where the implant is activated.

Referring to the RF envelope 930, it can be seen that during the time interval 912, the duty cycle of the power transfer apparatus of the external component goes to maximum power transfer six (6) times, and for most of those times, the maximum power lasts for 1 ms. The fourth power transfer, or more accurately, the fourth time that the power transfer goes to maximum in interval 912, lasts for two cycles. This can be because the power transferred during this maximum power transfer time transferred during the first cycle was not sufficient to raise the voltage of the implant above and/or to the target voltage (concomitant with the implant load remaining constant). That is, at the end and/or at a specified time during the first cycle, the system checks the voltage of the implant, and determines that the voltage is below the target voltage, and thus instructs the external component to remain at the maximum power transfer level for one more cycle, and this continues until a determination is made that the voltage of the implant meets and/or exceeds the target voltage, at which point the system instructs the external component to stop transferring power at the maximum voltage, which can thus correspond to transferring power at the minimum voltage. The bottom chart showing implant voltage versus time presents the target voltage 950 and the implant voltage 960 as measured for the time interval 912, and the additional time intervals. As can be seen, when a determination is made that the implant voltage 960 is below the target voltage 950, the external component is instructed to provide power to the implant at the maximum power level.

In some embodiments, with the caveat that some of these may have a very subtle and sometimes negligible impact on the link in the short term and/or medium term), the position of the external component relative to the implant component can shift, both with respect to lateral displacement and with respect to longitudinal displacement. Here, this can be a very subtle shift, which can be a result of simple inertia owing to movement of the recipient or even a recipient's pulse. Features which can change the link/impact the link and/or impact the load on the implant will be discussed below in greater detail.

Referring back to FIG. 9, it can be seen that during interval 912, there are six periods of low/minimum power transfer interleaved between the six periods of maximum/high power transfer. These periods of low/minimum power transfer also have varying lengths. Again, this embodiment of the power transfer regime is keyed off the implant voltage, and if the implant voltage is at and/or above a certain level, the target voltage, the external component transfers power at the lower level. Thus, as can be seen, there is a power transfer regime that is binary in nature. When the implant requires power, or, at least with respect to the time interval 912, when the implant needs power to maintain the target voltage, power is transferred from the external component at the maximum setting. When the requirements for voltage maintenance are met, maximum power transfer is halted and only minimum power transfer occurs.

As seen in FIG. 9, there is a second interval immediately after interval 912, time interval 914. During time interval 914, the load on the implant increases. This can be a result of an increasingly loud sound environment. By way of example only and not by way of limitation, this can correspond to a sound the volume of which is increasing rapidly in a linear manner. And note that while the increase in implant load shown in FIG. 9 is linear (and the decrease in implant load is also shown is linear), in other scenarios, the increase and/or decrease in load could be parabolic and/or exponential or otherwise could follow a curve. Regardless of why the implant load is increasing during time interval 914, it can be seen that for the roughly 20 ms that is the length of time interval 914, load on the implant is increasing, and that increase is in a linear manner. With reference to the RF envelope 930, it can be seen that there are seven (7) time periods where the external component goes to maximum power to transfer power to the implant, each of the time periods of high power transfer separated by a period of low power transfer. Further, it can be seen that these periods of high power transfer vary in length. This can be for any of the reasons detailed above and/or the reasons that will be further expanded upon below. With reference to the implant voltage versus time chart, it can be seen that as the implant voltage 960 falls below the target voltage 950, a period of maximum power transfer commences and the temporal period of the maximum power transfer is maintained until the implant meets and/or exceeds the target voltage, and then a period of low and/or minimum power transfer commences until the implant voltage falls below the target voltage.

And note that the sampling of the implant voltage occurs in a digital manner. Here, the implant voltage is sampled once every millisecond. In other embodiments, the implant voltage can be sampled at least once every two or three or 4 ms, or could be sampled faster than 1 ms (or less than these time periods). The implant voltage can be sampled for every cycle or can be sampled every two or three or four cycles or more. And note that the times that the implant voltage is sampled can vary depending on a given scenario. For example, during normal operation, the implant voltage can be sampled every cycle, but after a period of prolonged silence or otherwise after a period where the implant voltage has been maintained steady, the system could start sampling implant voltage every two or three or more cycles. Still, in some embodiments, the implant voltage is always sampled every cycle. This can have utilitarian value with respect to enabling the system to immediately meet the needs of the implant with respect to the implant load, or more accurately, when combined with the arrangement where the external component immediately applies maximum power with respect to the power transferred to the implant, the system can be enabled to meet the immediate needs of the implant with respect to implant load.

FIG. 9 also shows time interval 916 where the increase of implant load levels out and is constant albeit over twice as high as that which was the case during time interval 912. But as can be seen, the RF envelope shows that the length of time that the external component transfers power at the maximum power level at each of the maximum power level transfer time periods is longer than those of the time interval 914. This is the external component providing power to the implant at the higher implant load to maintain the implant at or above the target voltage. But there is more. Shown on FIG. 9 is dashed line 915. This represents a change to the overall system relative to that which is the case immediately precedent. Here, in this exemplary scenario, this represents the recipient changing a volume cochlear implant system (or the system automatically changing the volume), which change results in the implant load increasing beyond that which is directly attributable to the change in the environment that caused the linear increase of interval 914. Here, the increase in volume increases the required current level output by the electrodes of the cochlear implant. Alternatively, by way of example only, this could correspond to a change in the spectral makeup of the sound environment, where different electrodes of the electrode array are energized more so than that which was previously the case, which electrodes have higher impedances relative to the electrodes used to stimulate at the prior frequencies. Indeed, in an alternative embodiment, the beginning of time interval 914 can represent the beginning of the increase of volume of the cochlear implant, which increase is linear, and then time location 915 can correspond to an increase in loudness of an environment. In any event, as seen, the length of time that the external component applies the maximum power is longer after time location 915 then before time location 915. As seen, during time interval 916, the lengths of time of maximum power are about as long as the length of time of maximum power during time interval 914 after time location 915. In this exemplary scenario, this can be because the cochlear implant is set at the volume that caused the increase at time location 915 and/or because the environment is louder during this time interval than that which was the case in time interval 914. Also as can be seen, during time interval 916, the length of time of the periods of low power transfer are the shortest as compared to the prior two time intervals. The reasons for this can be seen when the RF envelope chart is compared to the implant voltage versus time chart as shown in FIG. 9. As seen, the increased load demand by the implantable component causes the implant voltage to drop to levels that are relatively low (after the increase to the level above the target voltage), and that drop occurs at a relatively steep rate relative to the occurrences prior to time interval 916. Indeed, as can be seen, the implant voltage increase rate from the beginning of each maximum power transfer period is lower during time interval 916 then that which was the case during time interval 912 and time interval 914. Also as can be seen, the implant voltage decrease rate after the completion of each of the maximum power transfer periods (the rate during the periods of minimum power transfer) is greater during time interval 916 than that which was the case during time intervals 912 and 914. This can be because the implant load is higher than that which was the case during the preceding time intervals, and thus the steeper the drop off and the more gradual the voltage increase (because more real time power is being consumed to evoke a hearing percept and thus less power is available to charge the capacitors, for example, thus slowing the rate of increase of the voltage of the implant.

Again, all of these scenarios are exemplary and are presented to simply explain how the power transfer regime according to the present embodiment can operate/functions.

FIG. 9 also shows time interval 918. Time interval 918 can be a time interval where, at the beginning, relative silence occurs, or otherwise the environment associated with time interval 912 returns. Here, the cochlear implant can be set at the same volume as that which was the case at the beginning of time interval 916 (and thus higher than that which was the case during time interval 912), but because of the relative silence, the implant is not stimulating and thus the load on the implant is the same as that which was the case during time interval 912. If for example, the environment during time interval 912 was an environment where the noise level was constant but not silent, and thus the implant was stimulating at that constant level, the drop off in implant load from time interval 916 to time interval 918 would not go all the way back to the same level as that which was the case during time interval 912, because the volume of the cochlear implant would be set higher than that which was the case during time interval 912. In any event, in the scenario under description utilized to explain the occurrence of the change in the implant load, at the beginning of time interval 918, there is silence, but then immediately, there is an increasing noise level, which thus increases the load on the implant because the implant is steadily increasing the current applied by the electrodes to convey the increase in ambient loudness to the recipient during time interval 918 (the loudness increases linearly).

Time interval 922 is a time interval of more rapidly increasing load on the implant. This can be due to the change in the environment or other reasons. But as seen, in the middle of time interval 922, a change occurs that more rapidly increases the implant load. The change is sufficiently rapid that for the remainder of the time interval, the external component delivers maximum power to the implant. At time interval 924, the implant load levels off at a very high level. Here, this can correspond to the implant delivering the maximum comfort level output from the electrodes for the sound frequencies to which it is exposed. For example, the ambient environment can be very loud, and thus the maximum output as set during fitting of the implant occurs during time. 924. As seen, the implant voltage effectively remains below the target voltage for the entire period of time interval 924. The implant voltage is low but does not reach the reset level, which can be for example, 3 V. Time. 924 ends with the ambient sound reducing, and thus the electrodes no longer stimulate at the maximum comfort level. The implant voltage thus shoots up above the target level voltage, and then the power transfer regime transitions the power output to the minimum power output as can be seen. This thus begins time interval 926, which is an interval of decreasing implant load on the implant. As seen, as the implant load decreases, the time intervals between the maximum power periods begin to be further spaced out, and the maximum power periods begin to be shortened in length for the most part. As seen, the implant voltage increases rapidly between the given updates at a rate more rapid than that which was the case during time interval 916, by way of comparison only. Thus, the implant voltage increases above the target voltage more than that which was the case in interval 916. And because the load is relatively low, the time above the target voltage is also longer relative to that which was the case during interval 916.

By way of example, with the caveat that some of these may have a very subtle and sometimes negligible impact on the link in the short term and/or medium term. Indeed, the skin flap thickness (the distance between the implanted coil and the external coil) can change relatively immediately for a number of physiological reasons, such as, for example, movement of the head that tensions and/or bunches up the skin at that location, and/or owing to movement of the recipient where the inertia of the external coil (or even the implanted coil for that matter), changes the lateral and/or longitudinal displacement of the coils. Moreover, things such as temperature or other conditions can change the implant tuning and/or the external coil tuning. In an exemplary embodiment, again with respect to the caveat above, which will be expanded on momentarily, the impedance of the recipient's tissue could have changed since the prior power transfer (although again, this may often be negligible in the short term because impedance of tissue may not change quickly enough to impact the link over the course of a few or 5 or 10 milliseconds, the short term), the required stimulation currents could change, the environmental sound conditions could change and implant electronic load variations can change. Granted, these things can impact the link in the medium term (e.g., more than a second or two) in a greater amount than that which is recoverable in a millisecond (e.g., the average length of maximum power might be longer for power transfer during the medium term), and these can also impact the link in the long term (e.g., more than 5 minutes). But it is possible for these phenomena to impact the link in the short term, however minor. A scenario that may change the power requirements of the implant in the medium-term and/or in the long-term can be the stimulation strategy applied by the cochlear implant system. Also, the required stimulation current can drastically affect the power requirements of the implant. Indeed, current output is a large if not the singularly most power intensive feature of a cochlear implant. Corollary to this is the sound environment in which the cochlear implant is located. The louder/more complex the sounds, the greater the load of the implant. All these things can change rapidly. Some additional factors that could result in large implant load transients (that can cause the implant voltage to drop quickly, where the teachings herein can react quickly to keep the voltage from dropping as much as it otherwise would drop, and thus avoid reset, all by example) can be sporadic communications on wireless links, periodic diagnostics that run from time to time in the implant, access to non-volatile memories for the purposes of data logging or reading of data (e.g., maps, firmware etc.) and periodic functions such as battery management, data integrity checks, watchdogs, etc.

And note that there are corollaries for a retinal implant, such as bright light (analogous to loud sounds impacting load on the implant), load variations, etc. Moreover, skin flap thickness, implant tuning and external coil tuning, for example, can affect the power transfer with respect to a retinal prosthesis.

In view of the above, in an exemplary embodiment, there is a device, such as a hearing prosthesis including a cochlear implant, or some other type of sensory prostheses, such as a retinal implant, that includes an external component, such as a behind-the-ear device including a signal processor and a microphone, or a light processor and a light capture device, to which is connected a transcutaneous radiofrequency inductance communication coil. This is shown in FIG. 1 above. This external component of the prosthesis is configured to provide power (transcutaneously) to an implanted device implanted in a human, such as by way of example only and not by way of limitation, the implantable portion of a cochlear implant electrode array. This implantable component also includes a radiofrequency inductance communication coil. In an exemplary embodiment, the implanted component can transmit data to the external component. More specifically, the external component can transmit power and/or data to the implant by way of a forward link, and the external component can, at least in some embodiments, transfer at least data from the implant to the external component via a backward link. In some embodiments, the external component pauses transmission in a manner that is synchronized with a logic circuit of the external component or otherwise pauses transmission in a manner that can be detected or where the external component notifies the implant that the pause will commence or has commenced, and then the implant can communicate to the external component.

The implanted device is a power consuming device that may or may not include a battery (but will include capacitors in many embodiments). In some embodiments, the implanted device cannot operate for more than a few seconds (if that) without the external component providing power thereto (when the capacitors sufficiently discharge without recharge). In an exemplary embodiment, the implantable component, or more accurately, the electronics of the implantable component, have a voltage level during operation, which voltage level will vary with load on the electronics and the amount of power being transferred to the implant by the external component. The implantable component is designed to operate within a minimum and maximum voltage of the electronics thereof. In this regard, the implant is often designed so that the minimum voltage is an amount greater than the implant reset voltage. The implant reset voltage is a voltage where, if the implantable component falls below this voltage, the implantable component reset itself, which can be effectively turning the implant off and then back on. Thus, in some embodiments, the implant reset voltage can be 1 or 1.5 or 2 or 2.5 or 3 or 3.5 or 4 V or more or less for that matter, or any value or range of values therebetween in 0.05 Volt increments. The minimum voltage of the operating range of the implantable component can be 0.25 or 0.5 or 0.75 or 1 or 1.25, or 1.5 or 1.75 or 2 V or more or less, or any value or range of values therebetween in 0.05 Volt increments, above the reset voltage. The maximum voltage of the operating range can be 0.25 or 0.5 or 0.75 or 1 or 1.25, or 1.5 or 1.75 or 2 V or more or less, or any value or range of values therebetween in 0.05 Volt increments, below the so-called manufacturing voltage or shunt voltage, which can be 7 or 7.5 or 8 or 8.5 or 9 or 9.5, or 10 or 10.5 or 11 or 11.5 or 12 or 13 or 14 volts or more or any value or range of values therebetween in 0.05 Volt increments.

It is between (and inclusive) the minimum operating voltage in the maximum operating voltage that the target voltage is located. In an exemplary embodiment, the target voltage will be set at 5.5 volts, by way of example only. But as will be described in greater detail below, the system can be configured to adjust the target voltage depending on the performance needs of the implant and/or the load to which the implant is subjected. For the moment, it will be assumed that the implant operates at a set 5.5 voltage.

The external component is configured to provide a plurality of levels of power output at any instant in time, which outputted power is to be received by the implant so as to maintain the implant voltage at the given target voltage. In an exemplary embodiment, the external component is configured so that a level of power output to the implanted device is dynamically varied. This dynamic variation is based on data based on a load of the implanted device. This data can be obtained via the backward link telemetry as noted above. Alternatively, and/or in addition to this, this data can be obtained by analyzing properties of the current flow and/or voltage of the coil of the external component, where such properties can be utilized as latent variables to estimate or otherwise evaluate the voltage of the implant at any given point in time.

In the exemplary embodiment detailed above, the external component is configured to provide the power output in a digital binary manner with respect to the level of power output thereof. It is noted that power output does not include the absence of power output. Accordingly, the aforementioned digital binary manner does not include zero power output as one of the two components. Instead, the digital binary manner includes a first output level and a second output level. As briefly detailed above, the first output level can be a maximum power output of the external component, and the second output level can be a minimum power output of the external component. These minimum and maximum power output components can be set accordingly for the transcutaneous transfer power regime that is being applied. In an exemplary embodiment, the first output level can be a value at or above 75, 80, 85, 90, or 95% or any value or range of values therebetween in 1% increments of a maximum possible power output of the external component. For example, this would be the maximum power output in the absence of all software and/or firmware constraints (where software could limit the output—indeed, in an exemplary embodiment, that is how the first output level is set—the external component could output more, but it is prevented by software and/or firmware). In an exemplary embodiment, the first power level can be at or above the maximum steady state load of the implant (e.g., within 1, 1.5, 2, 2.5, 3, 3.5, 4, 4.5, 5, 5.5, 6, 6.5, 7, 7.5, 8, 8.5, 9, 9.5, or 10% of the maximum steady state load (providing that it is either at that load or above).

In an embodiment, the second output level can be a value at or below 60, 55, 50, 45, 40, 35 or 30% or any value or range of values therebetween in 1% increments of the maximum possible power output of the external component. In an embodiment, the second output level can be at or above the minimum steady state load of the implant (e.g., within 1, 1.5, 2, 2.5, 3, 3.5, 4, 4.5, 5, 5.5, 6, 6.5, 7, 7.5, 8, 8.5, 9, 9.5 or 10% of the maximum steady state load (providing that it is either at that load or below). In some embodiments, the second output level can be at or below the minimum steady state load of the implant (and again within any of the just mentioned ranges).

Thus, with reference to the RF envelope of FIG. 9, the high values could be 90% of the maximum possible power output of the external component and the low values could be 50% of the maximum possible power output of the external component. Still with reference to the RF envelope of FIG. 9, the high values could be values within 4 percent of the maximum steady state load of the implanted component and the low values could be within 4 (or 5—the values need not be the same) percent of the minimum steady state load of the implanted component.

In an embodiment, the maximum possible power output of the external component can be the power output obtainable if control componentry of the external component that enable the digital binary mannered dynamic variation of power output level was eliminated.

In an embodiment, the digital binary manner comprises a maximum power output and a lowest level power output. In an embodiment, this is set for the given power transfer regime.

In an embodiment, the external component is configured to dynamically vary the level of power output to the implanted device in the digital binary matter irrespective of the load of the implant. In an embodiment, the external component is configured to dynamically vary the level of power output to the implanted device in the digital binary matter at least at values of load of the implant between (inclusive) any values or range of values spanning 10, 15, 20, 25, 30, 35, 40, 45, 50, 55, 60, 65, 70, 75, 90, 85, 90, 95, or 100% of the maximum operating load of the implant. In an embodiment, the external component is configured to dynamically vary the level of power output to the implanted device in the digital binary matter at least at values of voltage of the implant between (inclusive) the minimum operating and the maximum operating voltage and/or at or above the reset voltage to at or below the shunt voltage or any value or range of values therebetween in 0.01 volt increments.

In an embodiment, the external component is configured to immediately (e.g., as shown in FIG. 8) and/or effectively immediately increase an output power level by at least 30, 35, 40, 45, 50, 55, 60, 65, 70, 75, 80, 85, 90, 95, 100, 105, 110, 115, 120, 125, 130, or 135% or more, or any values or range of values therebetween in 1% increments (e.g., when going from the low power output to the high power output) and immediately and/or effectively immediately decrease the output power level by at least 20, 25, 30, 35, 40, 45, 50, 55, 60, 65, 70, 75, or 80% or more, or any values or range of values therebetween in 1% increments (e.g., when going from the high power output to the low power output). In an exemplary embodiment, the increases and/or decreases are set and are always the case for all periods of operation of the device. In an embodiment, the increases and/or decreases are the case for at least 5, 10, 15, 20, 25, 30, 35, 40, 45, 50, 60, 70, 80, 90, 120, 150, 200, 250, 300, 350, 400, 450, 500, 600, 700, 800, 900 or 1000 continuous seconds or minutes of operation (e.g., no reset of the implant) where there are at least 10, 15, 20, 25, 30, 35, 40, 45, 50, 60, 90, 120, 150, 200, 250, 300, 350, 400, 450, 500, 600, 700, 800, 900, 1000, 1250, 1500, 1750, 2000, 2500, 3000, 3500 or 4000 or more (or ten, 100 or 1000 times these amounts) increases and/or decreases during the time period.

In an embodiment, the digital binary manner comprises a first level and a second level. While embodiments above have focused on these levels being the maximum and the minimum, in other embodiments, this need not be the case. These levels can be, in an embodiment, levels that have values (or fall within a range of values that do not vary more than 0.1, 0.2, 0.3, 0.4, 0.5, 0.75, 1, 1.25, 1.5, 1.75, 2, 2.25, 2.5, 2.75, 3, 3.5, 4, 4.5, 5, 5.5, 6, 6.5, 7, 7.5, 8, 9, 10, 11, or 12%, or any value or range of values therebetween in 0.01% increments from the lowest value of the range) that correspond, respectively to the longest period of time of a given output level and a second longest period of time of a given output level if different than the longest period of time (if the same (an unlikely event), then the first level and the second level can be arbitrarily applied). In fact, the first level and the second level can be the first mode and the second mode (as in mean, median and mode averages) of a period of use of the implant. In an exemplary embodiment, the period of use of the implant can extend over any of the time periods detailed herein and/or can be different, such as by way of example only and not by way of limitation, at least and/or more than 1, 1.5, 2, 2.5, 3, 3.5, 4, 4.5, 5, 5.5, 6, 6.5, 7, 7.5, 8, 8.5, 9, 9.5, 10, 10.5, 11, 12, 13, 14, 15, 16, 17, 18, 19, or 20 hours, or any value or range of values therebetween in 0.1 hour increments of continuous operation of the implant (the implant is not reset during those periods). In an embodiment, the period of use of the implant can be measured over 1, 2, 3, 4, 5, 6, 7, 8, 9, 10, 11, 12, 13, 14, 15, 16, 17, 18, 19, 20, 21, 22, 23, 24, 25 contiguous days, or weeks, or months, or any value or range of values therebetween in 1 day increments (this is not contiguous/consistent operation-this is looking at the two power levels that have the longest period of time applied over that time, even though the device has been shut off/not been used during intervals within the survey period.

Thus, in an embodiment, there is a device where the digital binary manner comprises a first power output level and a second power output level different from the first power output level and the first power output level is a power level that, in totality of application during a continuous use of the external component (no resets for example) and/or continuous use of the external component to power the implant/keep the implant from resetting, is outputted at a longest period of time, and the second power output level is a power level that, in totality of application during the continuous use, is outputted at a second longest period of time. In an embodiment, instead of “continuous use,” the time period can be any of those herein. It can be the period of time between recharges of the external component or battery changes of the external component or the period of time when the external component is active and the external coil is in inductance communication with the implanted coil.

In an embodiment, one of the first power level or the second power level is lower than the other, and the other of the first power level or the second power level is at least 20, 25, 30, 35, 40, 45, 50, 55, 60, 65, 70, or 75% or any value or range of values therebetween in 0.1% increments higher than the one of the first power level or the second power level.

In an embodiment, the power level associated with the longest period of a power level will be the minimum/low power output. In an embodiment, the power level associated with the longest period of time or the second longest period of time will be the maximum/high power output. In an embodiment, the period of high/maximum power output will be collectively, during one or more of the time periods/periods of survey herein, excluding the period of the minimum/low power, longer than the next longest and/or longer than all other periods (again not including the low/minimum power output) by at least 20, 25, 30, 35, 40, 45, 50, 60, 70, 80, 90, 100, 125, 150, 175, 200, 225, 250, 275, 300, 350, 400, 450, 500, 550, 600, 700, 800, 900, 1000, 1250, 1500, 1750, 2000, 2250, 2500, 3000, 4000, 5000, 6000, 7000, 8000, 9000, 10000, 12500, 15000, 20000, 25000, 30000, 35000, or 40000% or more, or any value or range of values therebetween in 1% increments.

As seen above, embodiments can include transitioning from the minimum power output to the maximum power output over a single cycle (which in some embodiments is a millisecond) and/or vice versa. Embodiments can include transitioning from the minimum power output to the maximum power output within 0.5, 1, 1.5, 2, 2.5, 3, 3.5, 4, 4.5, 5, 5.5, 6, 6.5, 7, 7.5, 8, 8.5, 9, 9.5, 10, 11, 12, 13, 14, or 15 ms, or any value or range of values there between in 0.05 ms increments and/or vice versa for the transition from maximum power to the minimum power (and the values need not be the same, the transition up can happen faster than the transition downward or vice versa). The completed transition can occur within 1, 2, 3, 4, 5, 6, 7, 8, 9, 10, 11, 12, 13, 14, or 15 cycles.

Thus, as can be seen from the above, at least some embodiments include rampless transitioning from one power level to the other power level and some embodiments include steep ramps of transition.

Embodiments in view of the above include an adaptive power transfer system, wherein, in some examples of such, the adaptive power transfer system adapts to different load on the implant to keep the implant at a given voltage (for example—other “goals”/operating parameters can be used as a basis to trigger the adaptation of the system).

In an exemplary embodiment, the ramp up time from the minimum/low power transfer level to the high power/maximum power level is less than and/or equal to 0.1, 0.2, 0.3, 0.4, 0.5, 0.6, 0.7, 0.8, 0.9, 1.0, 1.25, 1.5, 1.75, 2, 2.25, 2.5, 2.75, 3, 3.5, 4, 4.5 5, 6, 7, 8, 9, 10, 11, 12, 13, 14, or 15% of the total time of the immediately preceding lower power minimum power transfer time period and/or the total time of the immediately preceding high power/maximum power transfer time period. In an embodiment, this is the case for at least 3, 4, 5, 6, 7, 8, 9, 10, 11, 12, 13, 14, 15, 16, 17, 18, 19, 20, 21, 22, 23, 24 25, 26, 27, 28, 29, or 30, or any value or range of values therebetween in 1 value increments transitions from low to high power over a period of time not extending past 1, 2, 3, 4, 5, 6, 7, 8, 9, 10, 11, 12, 13, 14, 15, 16, 17, 18, 19, or 20 seconds, or any value or range of values therebetween in 1 second increments. This can be the case for 70 or 75 or 80 or 85 or 90 or 95% or more of all such transitions during a day's use of the cochlear implant. This can also be the case in reverse for transitions from the maximum power to the minimum power.

FIG. 9A shows exemplary ramp ups and ramp downs. As seen, the first ramp up, which spans two cycles (2 ms) would not meet the requirement that it be less than 10% of the time of the proceeding max power, but the remaining ramp up would meet that (and the preceding minimum power), and the first ramp down would meet the 10 percent requirement for both the preceding high power and the proceeding low power.

In an exemplary embodiment, during ramp up or ramp down, there is not a power output level that is the same for two cycles and/or for 3 or 4 or 5 or 6 or 7 or 8 or 9 or 10 cycles during the ramp up or ramp down period. In an embodiment, the maximum power level output is maintained for at least and/or equal to 4, 5, 6, 7, 8, 9, 10, 11, 12, 13, 14, or 15 cycles or any value or range of values therebetween in 1 cycle increments when there is a precedent ramp up and/or proceeding ramp down. Herein, cycles can be 1 ms, or longer or shorter, as detailed herein. Reference to a cycle corresponds to an alternate disclosure of the temporal period associated therewith in accordance with the various temporal periods for the cycles disclosed herein for purposes of textual economy and vis-a-versa.

Thus, it can be seen that embodiments that dynamically vary the level of power output in a digital binary manner can include ramp up and ramp down periods interleaved between the binary power outputs. As noted above, in some embodiments, these ramp ups and ramp downs are temporally very limited, so limited that some can be effectively immediate.

As noted above, embodiments are such that the external component includes an inductance power transfer coil and the external component is configured to digitally dynamically vary a duty cycle of engagement of the coil to dynamically vary the level of power output to the implanted device in the digital binary manner.

In an embodiment, there is a device, such as any of the devices detailed above, that includes for example an external component of a prosthesis configured to provide power to an implanted device implanted in a human, wherein the external component is configured so that a principal power varying regime is based on varying power output to the implanted device by varying lengths of temporal periods of continuous maximum power output. The periods of continuous maximum power are those maximum power periods shown in FIG. 8, which are separated by periods of minimum power output, which happened to be continuous as shown. By “principal power varying regime,” it is meant that this is a design power regime that causes the external component to operate accordingly in a purposeful manner. This is opposed to a scenario where circumstances, such as the environment, simply cause the external component to operate where there are varying lengths of continuous maximum power output. Put another way, a principal power varying regime is not something that permits the features thereof to happen, but one that causes the features thereof to happen. Indeed, this is the case when during another operating regime, these features may not be present. By way of example only and not by way of limitation, the power varying regime of the arrangement of FIG. 7 does not have a principal power varying regime that is based on varying power output to the implanted device by varying lengths of temporal periods of continuous maximum power output. Instead, the principal power varying regime thereof is based on varying power output to the implanted device by increasing and/or decreasing various levels of power output in a discrete manner. Granted, the length of time of a possible maximum power output could be varied, but that is not the principal of operation of that power varying regime.

In an embodiment, the system is configured to implement a plurality of principal power varying regimes that are different from each other. For example, the external component could be configured to implement a power regime that operates according to the embodiment of FIG. 7, and also a power regime that operates according to the embodiment of FIG. 8. In an embodiment, a user of the external component can control the external component to switch from one power regime to the other power regime. In an exemplary embodiment, this can be done automatically depending on the load and/or the settings of the system. By way of example only and not by way of limitation, if the implant transitions from monopolar stimulation to multipolar stimulation, the power needs of the implant can increase by a factor of two or three or more. In an exemplary embodiment, the implantable component can transition to the principal power varying regime that varies no ranks of temporal periods of continuous maximum power output from the principal power varying regime that varies the output level of power according to the power needs of the implant. In an exemplary embodiment, the implantable component can transition automatically.

The above said, in an exemplary embodiment, the system can be a system where there is only one principal power varying regime, and that is the one that varies the length of temporal periods of continuous maximum power output. In an exemplary embodiment, the system can be a system that has in its memory stored two or more principal power varying regimes, but there is one that is activated. In an exemplary embodiment, a healthcare professional or the like must intervene to change the implemented principal power varying regime. Again, in at least some exemplary embodiments, the power regimes that are implemented are software and/or firmware based, and thus a device can be transition from one of the other otherwise a device can be modified, at least in some embodiments, to operate according to a given power varying regime.

It is noted that the principal power varying regimes can be different from one that varies the length of temporal periods and/or can include other features. By way of example only and not by way of limitation, a principal power varying regime can include the above noted binary maximum and minimum power transfer. And this principal power varying regime need not be mutually exclusive with the principal power varying regime that is based on varying power output by varying lengths of temporal periods of continuous maximum power output.

In an embodiment, the principal power varying regime further includes varying lengths of temporal periods of minimum power output. And in some embodiments, the principal power varying regime has respective periods of minimum power output interleaved with respective periods of maximum power output. In an embodiment, there can be ramp up and/or ramp down periods between these lengths of temporal periods of minimum and maximum power output. In some embodiments, the transition between the temporal periods of minimum power output and maximum power output can be immediate and/or effectively immediate. In an embodiment, the periods of minimum power output and maximum power output are contiguous with each other.

In at least some embodiments, the principal power varying regime increases power to the maximum power output in a rampless and/or effectively ramp less manner and/or decreases power to the minimum power output in a ramp less and/or effectively ramp less manner. Thus, embodiments can include a principal power varying regime that varies power output in a ramp less and/or effectively ramp less manner.

Embodiments can include a principal power varying regime that varies a ratio of respective temporal lengths of maximum power output to respective temporal lengths of minimum power output. In an exemplary embodiment, the ratios are ratios of two consecutive power transfers.

Consistent with the teachings detailed above, in an exemplary embodiment, the external component is configured to vary power output in the principal power regime based on data based on power load of the implanted device. This can be based on feedback from the implant via the backward link, or can be based on latent variables.

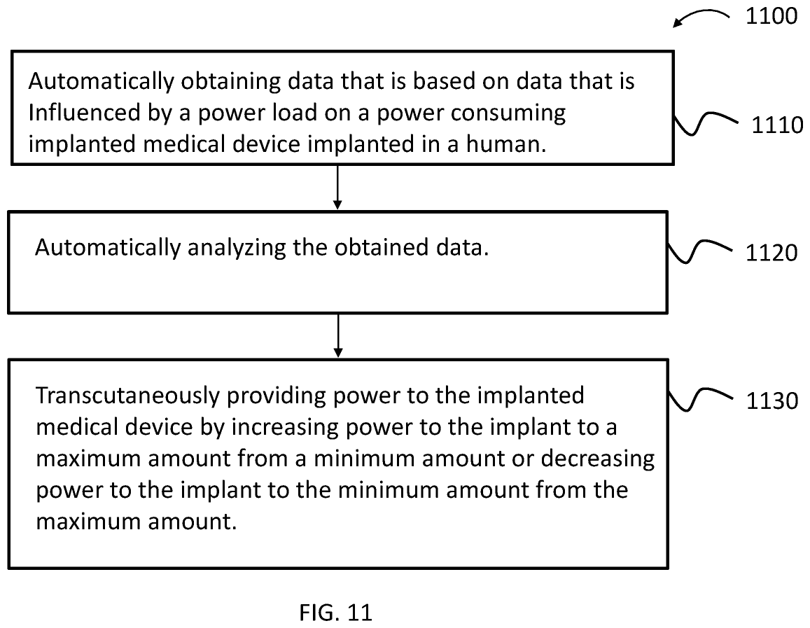

Embodiments include methods. FIG. 11 shows an exemplary flowchart for an exemplary method, method 1100, according to an exemplary embodiment. Method 1100 includes method action 1110, which includes the action of automatically obtaining data that is based on data that is influenced by a power load on a power consuming implanted medical device implanted in a human. For example, this could be obtaining a voltage level and/or a over or under indication that the implant is over the target voltage or under the target voltage. This could be an actual load value. This could be obtained by the backward link or could be obtained by latent variables or by evaluating a characteristic of the external coil for example. Any device, system, and/or method that can enable method action 1110 to be executed can be used providing that such enables the teachings herein. In an embodiment, method action 1110 is executed every cycle or every other cycle or every three cycles or by any algorithm that can have utilitarian value.

Method 1100 further includes method action 1120, which includes the action of automatically analyzing the obtained data. This can be done by the external component or can be done by the implantable component (or both). The result can be communicated to the external, or a simple command can be communicated to the external component. With respect to the latter, the implantable component can provide the results of this analysis via the backward link for example to the external component. This can be done automatically by a processor or by logic circuitry or the like. A lookup table could be utilized to analyze the obtained data. By way of example only and not by way of limitation, if the obtained data obtained in method action 1110 indicates that the voltage of the implant is lower than the target voltage, the analysis could indicate that the external component should apply or otherwise increase power transfer to the implanted component. And this leads to method action 1130, which includes the action of transcutaneously providing power to the implanted medical device by increasing power to the implant to a maximum amount from a minimum amount or decreasing power to the implant to the minimum amount from the maximum amount depending on the result of the analysis. In an embodiment, method action 1120 is executed every cycle or every other cycle or every three cycles or by any algorithm that can have utilitarian value.

In an embodiment, there is a method where, within a period 1, 2, 3, 4, 5, 6, 7, 8, 9, 10, 11, 12, 13, 14, 15 seconds, or minutes, or any value or range of values therebetween in 1 second increments (or any of the time periods detailed herein for purposes of textual economy), the actions of automatically obtaining and analyzing (actions 1110 and 1120) are executed X times, and Y results of the analysis of the obtained data is that an increase in a power level transcutaneously provided to the implanted medical device is needed, where X and Y (and they need not be the same) can be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 120, 140, 160, 180, 200, 250, 300, 350, 400, 450, 500, 550, 600, 700, 800, 900, or 1000 or more, or any value or range of values therebetween in 1 increment. (And note that the actions 1110 and 1120 can be executed more than X times—these are times that meet the method.) Further, in this embodiment, respective increases in the power level transcutaneously provided to the implanted medical device increase by at least 20, 25, 30, 35, 40, 45, 50, 55, 60, 65, 70, or 75% or any value or range of values therebetween in 1% increments within 1, 2, 3, 4, 5, 6, 7, 8, 9, 10, 11, 12, 13, 14, or 15 milliseconds, or any value or range of values therebetween in 1 ms increments.

In an embodiment, within a period corresponding to any of the above periods (at least as long in some embodiments), during which the actions of automatically obtaining and analyzing is repeatedly executed (any number of the times above for example), a load of the implanted medical device varies by at least 20, 25, 30, 35, 40, 45, 50, 55, 60, 65, or 70%, or any value or range of values therebetween in 1% increments upwards downwards (the values need not be the same each variation) at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 120, 140, 160, 180, 200, 250, 300, 350, 400, 450, or 500 times or more, or any value or range of values therebetween in 1 increment. A voltage of the implanted medical device and/or a mean and/or median voltage of the implanted device during that period does not deviate more than 2, 3, 4, 5, 6, 7, 8, 9, 10, 11, 12, 13, 14, 15, 16, 17, 18, 19, 20, 21, 22, 23, 24, 25, 26, 27, 28, 29, or 30%, or any value or range of values therebetween in 1% increments from the highest voltage during the period.

In an embodiment, prior to obtaining data based on data that is influenced by a power load, the implanted medical device experiences a beginning of a load transient that at least increases the load by 1.25, 1.5, 1.75, 2, 2.25, 2.5, 3, 3.5, 4, 4.5 or 5 times within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34 or 35 milliseconds or any value or range of values therebetween in 1 millisecond increments, and the obtained data is impacted by at least a portion of the load transient. For example, the voltage of the implant could decrease below the target, and thus the data based on data that is influenced by a power load on a per consuming implanted medical device could be the voltage of the implant. Alternatively, and/or in addition to this, it could be an indication that the voltage of the implant is below the target voltage. Raw load values can be the data on which the data is based. Other data can exist. In an embodiment, the action of transcutaneous providing power provides sufficient power that within 1, 2, 3, 4, 5, 6, 7, 8, 9, 10, 11, 12, 13, 14 or 15 milliseconds or any value or range of values therebetween in 1 millisecond increments of the completion of the transient, a voltage of the implanted medical device is returned to a value that is within 10, 9, 8, 7, 6, 5, 4, 3, 2 or 1% or any value or range of values therebetween in 0.1% increments of the value just before commencement of the load transient. In an embodiment, a voltage of the implanted medical device does not deviate from the target voltage or the voltage just before the transient by more than 40, 35, 30, 25, 20, 15, 10, 9, 8, 7, 6, 5, 4, 3, 2 or 1% or any value or range of value therebetween during the transient. In an embodiment, the mean and/or median deviation from the target voltage or the voltage just before the transient is no more than 30, 25, 20, 15, 10, 9, 8, 7, 6, 5, 4, 3, 2 or 1% during the transient.

By way of example, if the implant is consuming 5 mW during a first time interval, and then the load of the implant increases to 25 mW (which could happen if the stimulation strategy abruptly changes from monopolar to multipolar stimulation). This load increase could occur in less than 5 ms. A voltage of the implant just before the increase in load might be 5.7 volts. An embodiment is such that the voltage of the implant does not drop below 3.42 volts (a 40% drop).

In an embodiment, the power consuming implanted medical device has a voltage operating range that has a lower limit above a reset voltage and an upper limit below a shunt voltage, and prior to obtaining data based on data that is influenced by a power load, the implanted medical device experiences a beginning of a load transient that increases the load by any of the above noted amounts within any of the above noted periods, which at least increased load (e.g., quadrupled load) is present for at least 0.5, 1, 1.5, 2, 2.5, 3, 3.5, 4, 4.5, 5, 6, 7, 8, 9, 10, 11, 12, 13, 14, 15, 20, 25, 30, 35, 40, 45, or 50 seconds or more, or any value or range of values therebetween in 0.1 second increments, and the voltage of the implanted medical device remains in the operating range for that time period.

In an embodiment, the implanted medical device is an implantable component of a partially implantable cochlear implant, the action of transcutaneous providing power to the implanted medical device is executed by an external component of the partially implantable cochlear implant, the implanted medical device operates at maximum load for at least 0.5, 1, 1.5, 2, 2.5, 3, 3.5, 4, 5, 6, 7, 8, 9, 10, 11, 12, 13, 14, or 15 seconds, or any value or range of values therebetween during which the actions of automatically obtaining and analyzing is repeatedly executed (any number of the times above for example). In this embodiment, current output from electrodes of the implantable component equal set current values for all ambient sound captured during the period. In an embodiment, the current output for electrodes of channels for frequencies below 500, 1,000, 1500, 2,000, 2,500 or 3,000 Hz or any value or range of values therebetween in 1 Hz increments is at the comfort level or at least halfway or at least three/quarters of the way between the threshold level and the comfort level for at least 25, 30, 35, 40, 45, 50, 55, 60, 65, 70, 75, 80, 85, or 90% of the electrodes thereof during the time period of maximum load. In an embodiment, the cochlear implant is a fully implantable cochlear implant.

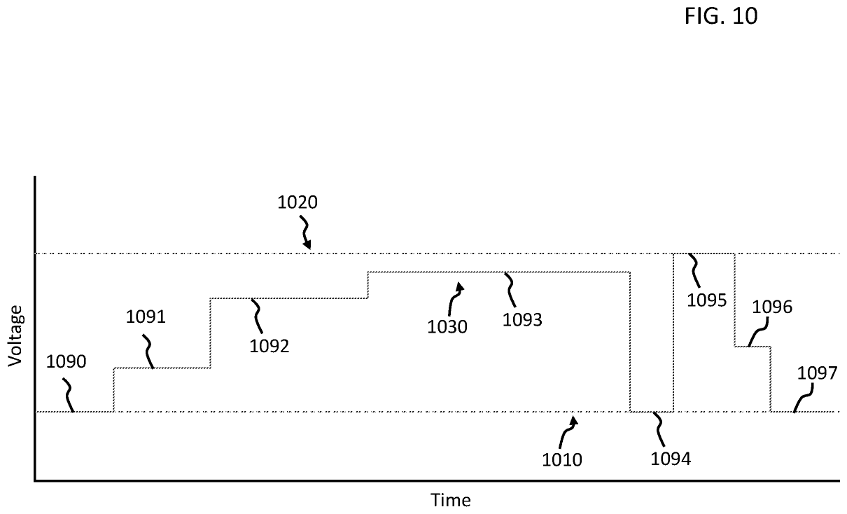

Embodiments can include adjusting the target voltage so as to provide for more of a “gap” between the lower operating range (and thus the reset) and the target voltage. This can reduce efficiency, but if managed properly, the reduction of the efficiency can be more limited. FIG. 10 shows an exemplary scenario where the target voltage 1030 is adjusted over time. Line 1010 corresponds to the set baseline target voltage. This is the target voltage that is ideal or otherwise the target voltage that the implant will have during most if not substantially all of its operating life. This target voltage can be the lower limit of the operating voltage noted above, or can be a voltage that is above the lower limit of the operating voltage. If the lower limit of the operating voltage is set sufficiently above the reset voltage, the set baseline target voltage at line 1010 can be the lower limit of the operating voltage, because there will be sufficient flexibility so that if the voltage drops below the lower limit operating voltage, for the brief period of time until the teachings detailed herein can raise the voltage, the drop will not reach the reset voltage because the teachings detailed herein can step in rather swiftly to slow and otherwise halt the decrease in target voltage. In this regard, in an exemplary embodiment, there is an operation of the implant over any one or more of the operating time periods detailed above where the deviation in the downward direction from the target voltage is never more than 1, 1.5, 2, 2.5, 3, 3.5, 4, 4.5, 5, 6, 7, 8, 9, 10, 11, 12, 13, 14, 15, 16, 17, 18, 19, 20, 21, 22, 23, 24, 25, 26, 27, 28, 29 or 30% or any value or range of values therebetween in 0.1% increments, even at the maximum possible load output of the implant (maximum current (comfort level current) at multipolar stimulation for example), and the below target voltage situation is never the case for more than 1, 1.5, 2, 2.5, 3, 3.5, 4, 4.5, 5, 6, 7, 8, 9, 10, 11, 12, 13, 14, 15, 16, 17, 18, 19, 20, 21, 22 or 23 ms, or any value or range of values therebetween in 1 ms increments, for the set baseline target voltage in some embodiments, and for other adjusted target voltages in other embodiments.

And with respect to the adjusted target voltages, again referring to FIG. 10, the system can be configured to adjust the target voltage according to the real time implant power needs. More particularly, as can be seen, the implant voltage is initially set at voltage 1090, which is the lowest voltage setting (line 1020 representing the maximum operating voltage, which is a level sufficiently below the shunt voltage). During a first time interval, the time that the implant remains below the target voltage is within acceptable or otherwise design time frames. Then, new circumstances exist, and there is utilitarian value with respect to raising the target voltage to the level 1091 as shown. Now, even if the scenario of use of the implant is such that the implant voltage will be reduced below the target voltage, there is more “room” between the target voltage and the reset voltage relative to that which was the case when the target voltage was set at 1090. As can be seen, the target voltage could be raised even further to level 1092. This provides even more “room” between the new set target voltage and the reset voltage. Level 1093 provides even more room between the target voltage and the new set voltage. As seen, in the scenario of use, the load on the implant or otherwise the forecasted load on the implant drops off and thus the system reduces the target voltage back to the baseline target voltage represented by level 1094 on line 1010. Then, owing to further changing circumstances where a very very high load is to be applied to the implant, the implant can raise the target voltage from the baseline all the way to the maximum operating voltage shown is level 1095 lying on line 1020. This represents the maximum implant voltage in the operating range, which maximum is below the shunt voltage by a utilitarian amount. As situations change, the set voltage is reduced by more than 50% of the range between the maximum and the minimum voltage to level 1096, and then as situations further change, the target voltage is reduced to the baseline target voltage 1097 as shown. Thus, embodiments can utilize the teachings above with a dynamically varying target voltage. This can have utilitarian value with respect to providing the implant in a powered state that permits the maximum or otherwise the desired current to flow from the electrodes. This can be utilitarian with respect to maintaining a desired volume perceived by the recipient, where a reduction in current can be perceived as a reduction in volume. Put another way, by varying the target voltage and otherwise increasing the target voltage above the baseline, it can be further assured that the implant will have the sufficient voltage level to provide the desired or otherwise the requisite current to evoke a hearing percept.

It is noted that any method action disclosed herein and/or functionality corresponds to a disclosure of a non-transitory computer readable medium that has program there on a code for executing such method action providing that the art enables such.

An exemplary system includes an exemplary device/devices that can enable the teachings detailed herein, which in at least some embodiments can utilize automation. That is, an exemplary embodiment includes executing one or more or all of the methods detailed herein and variations thereof, at least in part, in an automated or semiautomated manner using any of the teachings herein. Conversely, embodiments include devices and/or systems and/or methods where automation is specifically prohibited, either by lack of enablement of an automated feature or the complete absence of such capability in the first instance.

It is further noted that any disclosure of a device and/or system detailed herein also corresponds to a disclosure of otherwise providing that device and/or system and/or utilizing that device and/or system.

In an exemplary embodiment, one or more of the devices and/or systems and/or subsystems, etc., disclosed herein, and variations thereof, include a processor or a computer chip or a logic chip, which processor can be a standard microprocessor supported by software or firmware or the like that is programmed to execute one or more of the actions and functionalities herein or which chip can be constructed to execute one or more of the actions and functionalities herein. The processor and/or chip can include input and/or output connections. By way of example only and not by way of limitation, in an exemplary embodiment, the microprocessor can have access to lookup tables or the like having data and/or can compare features of the input signal and compare those features to features in the lookup table, and, via related data in the lookup table associated with those features, make a determination about the input signal, and thus make a determination, etc. Numeric analysis algorithms can be programmed in the processors, etc., to implement the teachings herein.

It is noted that the teachings detailed herein can be implemented in any processor-based device that can enable the teachings herein. In an exemplary embodiment, a sensory prosthesis, such as a hearing prosthesis or a light prosthesis, can be modified by adjusting the circuitry or otherwise providing programming to a given processor so as to enable the teachings detailed herein.

It is also noted that any disclosure herein of any process of manufacturing other providing a device corresponds to a disclosure of a device and/or system that results there from. Is also noted that any disclosure herein of any device and/or system corresponds to a disclosure of a method of producing or otherwise providing or otherwise making such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

Any function or method action detailed herein corresponds to a disclosure of doing so an automated or semi-automated manner.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

1. A device, comprising:
an external component of a prosthesis configured to provide power to an implanted device implanted in a human, wherein the external component is configured so that a level of power output to the implanted device is dynamically varied, based on data that is based on a load of the implanted device, in a digital binary manner.

2. The device of claim 1, wherein:
the external component is configured to dynamically vary the level of power output to the implanted device in the digital binary matter irrespective of the load of the implant.

3. The device of claim 1, wherein:
the digital binary manner comprises a maximum power output and a lowest level power output.

4. The device of claim 1, wherein:
the digital binary manner comprises a first output level which is at or above 80% of a maximum possible power output of the external component and a second output level which is at or below 60% of the maximum possible power output of the external component, the maximum possible power output being a power output obtainable if control componentry of the external component that enable the digital binary mannered dynamic variation of power output level was eliminated.

5. (canceled)

6. The device of claim 1, wherein:
the external component is configured to effectively immediately increase an output power level by at least 60% and effectively immediately decrease the output power level by at least 35%.

7. The device of claim 1, wherein:
the external component includes an inductance power transfer coil; and
the external component is configured to digitally dynamically vary a duty cycle of engagement of the coil to dynamically vary the level of power output to the implanted device in the digital binary manner.

8. (canceled)

9. The device of claim 1, wherein:
the device is a cochlear implant.

10. (canceled)

11. A device, comprising:
an external component of a prosthesis configured to provide power to an implanted device implanted in a human, wherein the external component is configured so that a principal power varying regime is based on varying power output to the implanted device by varying lengths of temporal periods of continuous maximum power output.

12. The device of claim 11, wherein:
the principal power varying regime further includes varying lengths of temporal periods of minimum power output.

13. The device of claim 11, wherein:
the principal power varying regime has respective periods of minimum power output interleaved with respective periods of maximum power output.

14. The device of claim 11, wherein:
the principal power varying regime increases power to the maximum power output in an effectively rampless manner.

15. The device of claim 11, wherein:
the principal power varying regime varies power output in an effectively rampless manner.

16. The device of claim 11, wherein:
the principal power varying regime varies a ratio of respective temporal lengths of maximum power output to respective temporal lengths of minimum power output.

17. The device of claim 11, wherein:
the external component is configured to vary power output in the principal power regime based on data that is based on power load of the implanted device.

18-20. (canceled)

21. A method, comprising:
automatically obtaining data based on data that is influenced by a power load on a power consuming implanted medical device implanted in a human;
automatically analyzing the obtained data; and
transcutaneously providing power to the implanted medical device by increasing power to the implant to a maximum amount from a minimum amount or decreasing power to the implant to the minimum amount from the maximum amount depending on the result of the analysis.

22. The method of claim 21, wherein:
within a period of 1 minute, the actions of automatically obtaining and analyzing are executed ten times, and ten results of the analysis of the obtained data is that an increase in a power level transcutaneously provided to the implanted medical device is needed; and
respective increases in the power level transcutaneously provided to the implanted medical device increase by at least 50% within 5 milliseconds.

23. The method of claim 21, further comprising:
prior to obtaining data based on data that is influenced by a power load, the implanted medical device experiences a beginning of a load transient that at least quadruples a load within 10 milliseconds, the obtained data being impacted by least a portion of the load transient, wherein
the action of transcutaneous providing power provides sufficient power that within 10 milliseconds of the completion of the quadrupling of the load, a voltage of the implanted medical device is returned to a value that is within 5% of the value just before commencement of the load transient.

24. The method of claim 21, wherein:
within a period of 1 minute, during which the actions of automatically obtaining and analyzing is repeatedly executed, a load of the implanted medical device varies by at least 30% upwards and downwards at least 5 times; and
a voltage of the implanted medical device does not deviate more than 25% from the highest voltage during the period of 1 minute.

25. The method of claim 21, wherein:
within a period of 1 minute, during which the actions of automatically obtaining and analyzing is repeatedly executed, a load of the implanted medical device varies by at least 30% upwards and downwards at least 5 times; and
a voltage of the implanted medical device does not deviate more than 10% from the highest voltage during the period of 1 minute.

26. (canceled)

27. The method of claim 21, wherein:
prior to obtaining data based on data that is influenced by a power load, the implanted medical device experiences a beginning of a load transient that at least quadruples a load within 10 milliseconds, the obtained data being impacted by least a portion of the load transient, wherein
the action of transcutaneous providing power provides sufficient power that during the load transient, a voltage of the implanted medical device is not reduced by any more than 30% from the voltage of the implanted medical device just before the load transient.

28. The method of claim 21, wherein:
the power consuming implanted medical device has a voltage operating range that has a lower limit above a reset voltage and an upper limit below a shunt voltage;
prior to obtaining data based on data that is influenced by a power load, the implanted medical device experiences a beginning of a load transient that at least quadruples a load within 10 milliseconds, which at least quadrupled load is present for at least 3 seconds, and the voltage of the implanted medical device remains in the operating range for that at least 3 seconds; and
the implanted medical device is an implantable portion of a cochlear implant.

29-30. (canceled)
